SEQUENCE LISTING (12 sequences)

SEQ ID NO: 1 - protein, 39 aa, Heloderma suspectum
  MISC_FEATURE (39)..(39): amidated optionally
HGEGTFTSDLSKQMEEEAVRLFIEWLKNGGPSSGAPPPS

SEQ ID NO: 2 - protein, 39 aa, Artificial Sequence
  synthetic peptide
HGEGTFTSDLSKQLEEEAVRLFIEWLKNGGPSSGAPPPS

SEQ ID NO: 3 - protein, 37 aa, Artificial Sequence
  synthetic peptide
HGEGTFTSDLSKQLEEEAVRLFIEWLKQGGPSKEIIS

SEQ ID NO: 4 - protein, 28 aa, Artificial Sequence
  synthetic peptide
HGEGTFTSDLSKQMEEEAVRLFIEWLKN

SEQ ID NO: 5 - protein, 28 aa, Artificial Sequence
  synthetic peptide
HGEGTFTSDLSKQMEEEAVRLFIEWLKN

SEQ ID NO: 6 - protein, 32 aa, Artificial Sequence
  synthetic peptide
KCNTATCVLGRLSQELHRLQTYPRTNTGSNTY

SEQ ID NO: 7 - protein, 63 aa, Artificial Sequence
  synthetic peptide
HGEGTFTSDLSKQMEEEAVRLFIEWLKNGGGKCNTATCVLGRLSQELHRLQTYPRTNTGSNTY

SEQ ID NO: 8 - protein, 62 aa, Artificial Sequence
  synthetic peptide
HGEGTFTSDLSKQMEEEAVRLFIEWLKNXXKCNTATCVLGRLSQELHRLQTYPRTNTGSNTY

SEQ ID NO: 9 - protein, 74 aa, Artificial Sequence
  synthetic peptide
HGEGTFTSDLSKQLEEEAVRLFIEWLKNGGPSSGAPPPSGGGKCNTATCVLGRLSQELHRLQTYPRTNTGSNTY

SEQ ID NO: 10 - protein, 74 aa, Artificial Sequence; identical to SEQ ID NO: 9
  synthetic peptide

SEQ ID NO: 11 - protein, 72 aa, Artificial Sequence
  synthetic peptide
HGEGTFTSDLSKQLEEEAVRLFIEWLKQGGPSKEIISGGGKCNTATCVLGRLSQELHRLQTYPRTNTGSNTY

SEQ ID NO: 12 - protein, 72 aa, Artificial Sequence; identical to SEQ ID NO: 11
  synthetic peptide

(19) United States
(12) Patent Application Publication
(10) Pub. No.: US 2014/0066370 A1
(43) Pub. Date: Mar. 6, 2014

(54) Polypeptide Conjugate

(71) Applicants: Behrouz Bruce Forood, Carlsbad, CA (US); Soumitra S. Ghosh, San Diego, CA (US); James L. Trevaskis, Newton, MA (US); Chengzao Sun, San Marcos, CA (US); Odile Esther Levy, San Diego, CA (US); Lawrence J. D'Souza, San Diego, CA (US); Christopher J. Soares, La Jolla, CA (US)

(72) Inventors: Behrouz Bruce Forood, Carlsbad, CA (US); Soumitra S. Ghosh, San Diego, CA (US); James L. Trevaskis, Newton, MA (US); Chengzao Sun, San Marcos, CA (US); Odile Esther Levy, San Diego, CA (US); Lawrence J. D'Souza, San Diego, CA (US); Christopher J. Soares, La Jolla, CA (US)

(73) Assignee: Amylin Pharmaceuticals, LLC, San Diego, CA (US)

(21) Appl. No.: 13/511,201

(22) PCT Filed: Nov. 23, 2010
(86) PCT No.: PCT/US10/57890

§ 371 Date: Oct. 11, 2012

Related U.S. Application Data
(60) Provisional application No. 61/263,752, filed on Nov. 23, 2009.

(51) Int. Cl. C07K 14/605

(52) U.S. Cl. CPC C07K 14/605
USPC 514/7.2; 435/69.1; 514/7.3; 530/322; 530/324; 530/333; 530/362

(57) ABSTRACT

The disclosure provides Polypeptide Conjugates with multiple improved pharmacological and pharmacokinetic properties and their use in treating various diseases and conditions, such as diabetes and/or obesity.

### CROSS-REFERENCE TO RELATED APPLICATIONS

This application claims benefit of U.S. provisional patent application No. 61/263,752, filed Nov. 23, 2009, the entire contents of which are incorporated herein for all purposes.

### FIELD

Provided herein are Polypeptide Conjugates that have both GLP-1 receptor agonist and amylin mimetic activities with superior pharmacological properties and therapeutic methods for their use.

### BACKGROUND

Peptides and proteins play critical roles in the regulation of biological processes. Peptides, for example, play a regulatory role as hormones and inhibitors, and are also involved in immunological recognition. The significant biological role of peptides makes it important to understand their interactions with the receptors to which they bind.

Incretin peptides are hormones and peptide mimetics that cause an increase in the amount of insulin released when glucose levels are normal or particularly when they are elevated. The concept of the incretin effect developed from the observation that insulin responses to oral glucose exceeded those measured after intravenous administration of equivalent amounts of glucose. These incretin peptides have other actions beyond the initial incretin action defined by insulin secretion. For instance, they may also have actions to reduce glucagon production, increase satiety or reduce food intake, and delay gastric emptying. In addition, they may have actions to improve insulin sensitivity, and they may increase islet cell neogenesis—the formation of new islets.

Although many postprandial hormones have incretin-like activity, predominant incretin peptides include glucose-dependent insulinotropic polypeptide, also known as gastric inhibitory polypeptide (GIP), glucagon-like peptide-1 (GLP-1), and exendin peptides (which are non-endogenous incretin mimetics). GIP and GLP-1 both belong to the glucagon peptide superfamily. GLP-1 is secreted by specialized cells in the gastrointestinal tract and have receptors located on islet cells as well as other tissues. GLP-1 is secreted from the intestine in response to ingestion of nutrients, which results in enhanced insulin secretion. The insulinotropic effect of GLP-1 is dependent on elevations in ambient glucose. GLP-1 is rapidly inactivated by the ubiquitous enzyme dipeptidyl peptidase IV (DPP-IV). Exendin-4 binds the GLP-1 receptors on insulin-secreting cells, stimulates insulin secretion in the presence of glucose, and the peptide also suppresses glucagon, delays gastric emptying and increases satiety and reduces food intake. The use of the insulinotropic activities of exendin-4 for the treatment of diabetes mellitus and the prevention of hyperglycemia has been proposed (Eng, U.S. Pat. No. 5,424,286). However, unlike GLP-1, exendin-4 has a relatively long half-life in humans, and further has a stronger capability to stimulate insulin secretion at a lower concentration of exendin-4.

Another family of peptide hormones implicated in metabolic diseases and disorders is the amylin family of peptide hormones, e.g. amylin, and its analogs, e.g. pramlintide and davalintide. The amylin molecule has two post-translational modifications: the C-terminus is amidated, and the cysteines in positions 2 and 7 are cross-linked to form an N-terminal loop. The sequence of the open reading frame of the human amylin gene shows the presence of the Lys-Arg dibasic amino acid proteolytic cleavage signal, prior to the N-terminal codon for Lys, and the Gly prior to the Lys-Arg proteolytic signal at the CLAIMS-terminal position, a typical sequence for amidation by protein amidating enzyme, PAM (Cooper et al., Biochem. Biophys. Acta, 1014:247-258 (1989)). Amylin is believed to delay gastric emptying, and suppress glucagon secretion and reduces food intake, thus regulating the rate of glucose appearance in the circulation. It appears to complement the actions of insulin, which regulates the rate of glucose disappearance from the circulation and its uptake by peripheral tissues. These actions are supported by experimental findings in rodents and humans, which indicate that amylin complements the effects of insulin in postprandial glucose control by at least three independent mechanisms, all of which affect the rate of glucose appearance. In human trials, an amylin analog, pramlintide, has been shown to reduce weight or weight gain. Amylin mimetics may be beneficial in treating metabolic conditions such as diabetes and obesity. Amylin mimetics may also be used to treat pain, bone disorders, gastritis, to modulate lipids, in particular triglycerides, or to affect body composition such as the preferential loss of fat and sparing of lean tissue.

Metabolic diseases and disorders take on many forms, including obesity, diabetes, dyslipidemia, insulin resistance, cellular apoptosis, etc. Obesity and its associated disorders are common and very serious public health problems in the United States and throughout the world. Upper body obesity is the strongest risk factor known for type 2 diabetes mellitus, and is a strong risk factor for cardiovascular disease. Obesity is a recognized risk factor for diabetes, hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia (see, e.g., Kopelman, Nature 404: 635-43 (2000)). It reduces life-span and carries a serious risk of co-morbidities above, as well disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., Br. Med. J. 301: 835-7 (1990)). Obesity is also a risk factor for the group of conditions called insulin resistance syndrome, or “Syndrome X.” Obesity is currently a poorly treatable, chronic, essentially intractable metabolic disorder. A therapeutic drug useful in weight reduction of obese or overweight persons, with or without diabetes, could have a profound beneficial effect on health.

Diabetes is a disorder of carbohydrate metabolism characterized by hyperglycemia and glucosuria resulting from insufficient production or utilization of insulin. Diabetes severely affects the quality of life of large parts of the populations in developed countries. Insufficient production of insulin is characterized as type 1 diabetes and insufficient utilization of insulin is type 2 diabetes. However, it is now widely recognized that there are many distinct diabetes related diseases which have their onset long before patients are diagnosed as having overt diabetes. Also, the effects from the suboptimal control of glucose metabolism in diabetes gives rise to a wide spectrum of related lipid and cardiovascular disorders. Overwieght and obesity are a common serious co-morbidity with diabetes, and in some cases may lead to or increase the propensity to diabetes.

Dyslipidemia, or abnormal levels of lipoproteins in blood plasma, is a frequent occurrence among diabetics. Dyslipidemia is typically characterized by elevated plasma triglycerides, low HDL (High Density Lipoprotein) cholesterol, normal to elevated levels of LDL (Low Density Lipoprotein) cholesterol and increased levels of small dense, LDL (Low Density Lipoprotein) particles in the blood. Dyslipidemia is one of the main contributors to the increased incidence of coronary events and deaths among diabetic subjects. Epidemiological studies have confirmed this by showing a several-fold increase in coronary deaths among diabetic subjects when compared with non-diabetic subjects. Several lipoprotein abnormalities have been described among diabetic subjects.

Attempts to treat the multiple abnormalities associated with diabetes have prompted for the administration of several anti-diabetic medicaments in order to address these abnormalities in the different patients. Examples of anti-diabetic medicaments are proteins such as insulin and insulin analogues, and small molecules such as insulin sensitizers, insulin secretagogues and appetite regulating compounds, which are often administered in concert to address the potential multiple abnormalities.

There remains a need to develop polypeptides useful in the above described metabolic diseases, conditions, and disorders. Accordingly, it is an object of the present invention to provide polypeptides useful to treat the diseases and conditions described herein and methods for producing and using the polypeptides.

### SUMMARY

The present invention relates generally to novel, Polypeptide Conjugates with multiple pharmacological actions that allow their use as agents for the treatment and prevention of metabolic diseases and disorders which can be alleviated either by control of plasma glucose levels, insulin levels and/or insulin secretion and/or further by increasing weight loss, reducing body weight, maintaining weight, preventing weight gain, reducing food intake, increasing satiety, reducing appetite and/or improving body composition by being lean-sparing but fat wasting, or by a combination of the glucose/insulin control and the weight loss/food-intake control properties present in the Polypeptide Conjugates described herein. Such conditions and diseases include hyperglycemia and hyperglycemia-related conditions, diabetes and diabetes-related conditions, obesity and overweight, and conditions where both a glucose/insulin control related and a weight loss/food intake-related diseases or conditions are present. Further such conditions and disorders include, but are not limited to, hypertension, dyslipidemia, cardiovascular disease, eating disorders, insulin-resistance, obesity, and diabetes mellitus of any kind, including type 1, type 2, and gestational diabetes, and combinations thereof. The present multi-action compounds also find use in conditions where hyperglycemia is an important factor, often in the absence of overt diabetes, and further where overweight or obesity is present or may occur, as in for example, steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in Subjects with congenital or HIV-Associated Lipoatrophy or “Fat Redistribution Syndrome”, and in Metabolic Syndrome (Syndrome X).

It has now been unexpectedly discovered that an anti-diabetic Polypeptide Conjugate can have similar or improved glucose lowering effects and further, have improved weight loss and/or reduction of food intake effects, compared to exenatide, i.e. exendin-4, a GLP-1 receptor agonist approved for treating diabetes in the United States and Europe, and compared to previously known conjugate peptide constructs. The disclosure herein is based on this discovery. The Polypeptide Conjugates of the present invention provide at least two pharmacological actions, e.g. glucose/insulin control and weight/food intake control, as described herein, to provide superior therapeutic benefit, e.g. acting in a synergistic fashion to improve or normalize multiple metabolic functions and abnormalities.

Provided are Polypeptide Conjugates Compound 1A and Compound 2A described herein that comprise a GLP-1 receptor agonist peptide conjugated to an amylin mimetic peptide. The Polypeptide Conjugates exhibit agonism of exendin-4 at a GLP-1 receptor and exhibit agonism of davalintide (an amylin mimetic) at a C1a receptor to provide, at the least, desirable control of glucose and insulin with a superior control of weight loss and/or food intake compared to either exendin-4 or davalintide.

In some embodiments, a Polypeptide Conjugate described herein is superior to a corresponding reference compound, e.g. exenatide or davalintide, or a corresponding reference conjugate compound having a different GLP-1 receptor agonist component and/or a different amylin mimetic component, e.g. Compound 3A and Compound 7A described herein. In this context, the term “superior” refers to a variety of functional properties which could be weighed in the evaluation of a treatment for a disease or disorder. For example, the Polypeptide Conjugate described herein could require a lesser dose for maximal efficacy, for example 1×, 2×, 3×, 4×, 5×, or even less, than the corresponding reference compound. For further example, the Polypeptide Conjugate described herein could have higher potency, for example, 1.5×, 2×, 3×, 4×, 5×, 10×, 20×, 50×, or even higher potency. For further example, the Polypeptide Conjugate described herein could have a function not found to a significant extent in the reference compound.

It is further understood that Polypeptide Conjugates described herein include those that have been chemically derivatized. Such derivatized peptides include conjugation to one or more polymer moieties, such as polyethylene glycol (PEG) or fatty acid chains of various lengths (e.g., stearyl, palmitoyl, octanoyl, etc.), or by the addition of polyamino acids, such as poly-his, poly-arg, poly-lys, and poly-ala. Modifications can also include small molecules moieties, such as short alkyls and constrained alkyls (e.g., branched, cyclic, fused, adamantyl) and aromatic groups. The polymer moieties will typically have a molecular weight from about 500 to about 60,000 Daltons. The polymer may be linear or branched. Such derivatizations can take place at the N- or C-terminus or at a side chain of an amino acid residue, e.g. lysine epsilon amino group, aspartic acid, glutamic acid, cysteine sulfhydryl group, within the Polypeptide Conjugate. Alternatively, derivatization can occur at multiple sites throughout the conjugate polypeptide. To provide a site(s) for derivatization, substitution of one or more amino acids with, or addition of, a lysine, aspartic acid, glutamic acid or cysteine can be done. See for example U.S. Pat. Nos. 5,824,784 and 5,824,778, which are incorporated by reference herein.

In one embodiment the Polypeptide Conjugates can be conjugated to one, two, or three polymer moieties. Pegylating the Polypeptide Conjugates can improve their aqueous solubility, increase plasma half-life, reduce immunogenecity and/or improve oral uptake. In one embodiment the pegylated Polypeptide Conjugates comprises a lysine side chain to which is covalently attached via the lysine epsilon amino group a polyethylene glycol moiety, and in a further embodiment the total molecular weight of the PEG moiety is at least about 10,000 Daltons, at least about 20,000 Daltons, at least about 40,000 Daltons or at least about 60,000 Daltons. In one embodiment the molecular weight of the PEG chain(s) is greater than 10,000 and less than or equal to 60,000 Daltons.

The Polypeptide Conjugates can be used for therapeutic purposes (e.g., treat diabetes); for research purposes; and to produce GLP-1 receptor agonist compounds having improved GLP-1 receptor binding activity and/or improved in vivo glucose lowering activity and/or improved amylin mimetic activity and/or improved weight loss/food-intake control activity, such as derivatives having longer duration of action, increased solubility and/or reduced immunogenecity. The disclosure provides pharmaceutical compositions comprising therapeutically effective amounts of the Polypeptide Conjugate. The disclosure also provides methods for synthesizing the Polypeptide Conjugate.

The disclosure provides methods for treating diabetes; treating insulin resistance; treating postprandial hyperglycemia; lowering blood glucose levels; lowering HbA1c levels; stimulating insulin release; reducing gastric motility; delaying gastric emptying; reducing food intake; reducing appetite; reducing weight; treating overweight; and/or treating obesity in subjects in need thereof by administering therapeutically effective amounts of the Polypeptide Conjugate described herein. The disclosure also provides methods for treating hyperglycemia and hyperglycemia-related conditions, diabetes and diabetes-related conditions, obesity and overweight, and conditions where both a glucose/insulin control related and a weight loss/food intake-related diseases or conditions are present, in subjects in need thereof by administering therapeutically effective amounts of a Polypeptide Conjugate described herein. The disclosure also provides methods for treating hypertension, dyslipidemia, cardiovascular disease, eating disorders, insulin-resistance, obesity and diabetes mellitus of any kind, including type 1, type 2, and gestational diabetes, and combinations thereof, in subjects in need thereof by administering therapeutically effective amounts of the Polypeptide Conjugate described herein. The disclosure also provides methods for treating steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in subjects with congenital or HIV-Associated Lipoatrophy or “Fat Redistribution Syndrome”, and Metabolic Syndrome (Syndrome X), in subjects in need thereof by administering therapeutically effective amounts of the Polypeptide Conjugate described herein.

The Polypeptide Conjugate can be co-administered with another anti-diabetes and/or anti-obesity agent. By “co-administered” is meant administration two or more active agents as a single composition, simultaneously administered as separate solutions, or alternatively, can be administered at different times relative to one another, such as within 4, 8 or 12 hours of one another (for example to avoid interference with uptake of the second agent due to delay of gastric emptying effects of the Polypeptide Conjugate.) The exact ratio of the Polypeptide Conjugate relative to the second agent will be dependent in part as determined by the physician and the needs of the subject.

The Polypeptide Conjugate can be provided in a kit suitable for use by the subject, where the kit comprises instructions for use of the Polypeptide Conjugate.

### BRIEF DESCRIPTION OF THE FIGURES

FIGS. 1A and 1B: FIG. 1A is a graph depicting change in body weight in DIO (diet induced obese) rats administered Compound 2A or the other compounds in the amounts shown in FIG. 1A as described in the Examples. FIG. 1B is a graph depicting change in body weight in DIO rats administered Compound 1A or the compounds in the amounts shown in FIG. 1B as described in the Examples. Groups not sharing a superscript are significantly different from each other; p<0.05, e.g. the vehicle controls of FIGS. 1A and 1B are not significantly different from each other.

FIGS. 2A and 2B: FIG. 2A is a graph depicting total cumulative food intake for DIO rats administered Compound 2A or the other compounds in the amounts shown in FIG. 2A as described in the Examples. FIG. 2B is a graph depicting total cumulative food intake for DIO rats administered Compound 1A or the other compounds in the amounts shown in FIG. 2B as described in the Examples. Groups not sharing a superscript are significantly different from each other; p<0.05, e.g. the vehicle controls of FIGS. 2A and 2B are not significantly different from each other.

FIG. 3: FIG. 3 is a graph showing change in adiposity in DIO rats administered Compound 2A or the other compounds in the amounts shown in FIG. 3 as described in the Examples. Groups not sharing a superscript are significantly different from each other; p<0.05.

FIG. 4: FIG. 4 is a graph showing change in adiposity in DIO rats administered Compound 1A or the other compounds in the amounts shown in FIG. 4 as described in the Examples. Groups not sharing a superscript are significantly different from each other; p<0.05.

FIG. 5: FIG. 5 is a graph showing change in percent lean mass of DIO rats administered Compound 2A or the other compounds in the amounts shown in FIG. 5 as described in the Examples.

FIG. 6: FIG. 6 is a graph showing change in percent lean mass of DIO rats administered Compound 1A or the other compounds in the amounts shown in FIG. 6 as described in the Examples. *p<0.05 vs. vehicle.

FIGS. 7A and 7B: FIGS. 7A and 7B are graphs showing the measured plasma drug levels (PK property) correlated with weight loss (a PD property) at Day 14 (FIG. 7A) and at Day 28 (FIG. 7B) for Cmpd 1A, Cmpd 2A and Cmpd 3A from the chronic DIO rat studies described herein.

### DETAILED DESCRIPTION

“GLP-1 receptor agonist compounds” refer to compounds that elicit a biological activity of an exendin reference peptide (e.g., exendin-4) or a GLP-1(7-37) reference peptide when evaluated by art-known measures such as receptor binding studies, cAMP generation or in vivo blood glucose and/or insulin secretion assays as described herein, and by Hargrove et al, Regulatory Peptides, 141:113-119 (2007), the disclosure of which is incorporated by reference herein. GLP-1 receptor agonist compounds include, for example, native exendins, exendin analogs, native GLP-1, GLP-1 analogs, GLP-1(7-37), and GLP-1(7-37) analogs.

The term “exendin” includes naturally occurring (or synthetic versions of naturally occurring) exendin peptides that are found in the salivary secretions of the Gila monster. Exendins include the amidated forms, the acid form, the pharmaceutically acceptable salt form, and any other physiologically active form of the molecule. In one embodiment, the term exendin can be used interchangeably with the term “exendin agonist.”

“Exendin analog” refers to peptides, peptides containing amino acid substitutions, insertions, deletions or additions, peptide mimetics, and/or other modifications, and/or other chemical moieties, which elicit a biological activity similar to that of an exendin reference peptide (e.g., exendin-4), when evaluated by art-known measures such as receptor binding assays or in vivo blood glucose assays as described herein and by Hargrove et al, Regulatory Peptides, 141:113-119 (2007). Exendin analogs include the amidated forms, the acid form, the pharmaceutically acceptable salt form, and any other physiologically active form of the molecule. In one embodiment, the term exendin analog can be used interchangeably with the term “exendin agonist analog.”

Exenatide (exendin-4) is a 39 amino acid glucagon-like peptide-1 (GLP-1) receptor agonist currently indicated for the treatment of type 2 diabetes, and also exerts weight loss and other metabolic actions when administered to DIO rats. The sequence of exendin-4 follows: HGEGTFTSDLSKQMEEEAVRLFIEWLKNGGPSSGAPPPS-NH₂ (SEQ ID NO: 1), where —NH2 indicates the presence of a C-terminal amidated amino acid. Exendin-4 is also active in its free acid form. Hargrove et al, Regulatory Peptides, 141:113-119 (2007), reported an exendin-4 peptide analog that is a full-length C-terminally amidated exendin-4 peptide analog with a single nucleotide difference at position 14 compared to native exendin-4, and which is designated herein as Compound 4A. Hargrove reported that this exendin-4 analog Cmpd 4A is remarkably inferior to exendin-4 with respect to its delaying of gastric emptying, anti-obesity properties and half-life. The sequence of Cmpd 4A follows: HGEGTFTSDLSKQLEEEAVRLFIEWLKNGGPSSGAPPPS-NH2 (SEQ ID NO: 2). Another exendin-4 peptide analog is designated herein as Compound 5, which is a chimera of the first 32 amino acids of exendin-4 having amino acid substitutions at positions 14 and 28 followed by a 5 amino acid sequence from the C-terminus of a non-mammalian (frog) GLP1. Cmpd 5 has the following sequence: HGEGTFTSDLSKQLEEEAVRLFIEWLKQGGPSKEIIS (SEQ ID NO: 3).

Also known in the art is a truncated, biologically active form of exendin-4, exendin-4(1-28), which is designated herein as Compound 10 having the following sequence: HGEGTFTSDLSKQMEEEAVRLFIEWLKN (SEQ ID NO: 4); and its amide form designated Compound 10A having the sequence HGEGTFTSDLSKQMEEEAVRLFIEWLKN-NH2 (SEQ ID NO: 5).

Amylin is a peptide hormone co-secreted with insulin by pancreatic beta-cells after nutrient ingestion whose primary physiological roles involve the inhibition of feeding behavior and gastric emptying, and subsequently reduced body weight. Davalintide, also known as “AC-2307” is an amylin agonist reportedly useful in the treatment of a variety of disease indications. See WO 2006/083254 and WO 2007/114838, each of which is incorporated by reference herein in its entirety and for all purposes. Davalintide is a chimeric peptide, having an N-terminal loop region of amylin or calcitonin and analogs thereof, an alpha-helical region of at least a portion of an alpha-helical region of calcitonin or analogs thereof or an alpha-helical region having a portion of an amylin alpha-helical region and a calcitonin alpha-helical region or analog thereof, and a C-terminal tail region of amylin or calcitonin. The amino acid sequence of davalintide sequence is as follows: KCNTATCVLGRLSQELHRLQTYPRTNTGSNTY-NH2 (SEQ ID NO: 6), which is designated Compound 6A herein. It has been reported that for diet-induced obese (DIO) rats, weight loss induced by amylin and by Cmpd 6A administration is characterized by preferential loss of adipose mass preservation of lean mass.

Previously described conjugates of an exendin analog and an amylin mimetic include Compound 3A having the sequence HGEGTFTSDLSKQMEEEAVRLFIEWLKNGGGKCNTATCVLGRLSQELHRLQTYPRTNT GSNTY-NH2 (SEQ ID NO: 7) where the polypeptide is C-terminally amidated, and Compound 7A having the sequence HGEGTFTSDLSKQMEEEAVRLFIEWLKN(beta-A)(beta-A)KCNTATCVLGRLSQELHRLQTYPRTNTGSNTY-NH2 (SEQ ID NO: 8) where the polypeptide is C-terminally amidated and contains a beta-alanine, beta-alanine unnatural amino acid dipeptide linker. The above compounds comprise an active C-terminally truncated form of exendin-4, Compound 10A exendin-4(1-28) amide: HGEGTFTSDLSKQMEEEAVRLFIEWLKN-NH2 (SEQ ID NO: 5).

The present invention is based on the surprising result of the superior effects of specific Polypeptide Conjugates described herein which comprise specific exendin-4 peptide analogs covalently linked to davalintide Cmpd 6A. The Polypeptide Conjugates are Compound 1A and Compound 2A described herein, and their chemical derivatives, e.g. pegylated. The Polypeptide Conjugates exhibit agonism of exendin-4 at a GLP-1 receptor and exhibit agonism of davalintide (an amylin mimetic) at a C1a receptor to at the least provide control of glucose with a superior weight loss/food intake control compared to either exendin-4 or davalintide. The compounds have superior glucose control with surprisingly superior weight control as well as superior pharmacokinetic properties compared to known conjugates and the parent peptides. The Polypeptide Conjugates have a combination of glucoregulatory and weight-loss inducing GLP-1 receptor agonism with the fat-specific weight loss inducing amylin mimetic activity, but with superior properties, e.g. greater therapeutic efficacy, improved half-life, to either parent peptide or known conjugates of this type.

The term “Polypeptide Conjugate” with respect to the present invention refers to Cmpd 1A and Cmpd 2A, which are C-terminally amidated, and includes their acid form, their pharmaceutically acceptable salt forms, and any other physiologically active form of Cmpd 1A or Cmpd 2A, including chemically modified derivatives, e.g. pegylated or fatty acylated derivatization as to extend plasma half-life or improve oral uptake.

Compound 1 described herein has the following sequence HGEGTFTSDLSKQLEEEAVRLFIEWLKNGGPSSGAPPPSGGGKCNTATCVLGRLSQELH RLQTYPRTNTGSNTY (SEQ ID NO: 9). Its C-terminally amidated form is Compound 1A, a Polypeptide Conjugate of the present invention, having the sequence HGEGTFTSDLSKQLEEEAVRLFIEWLKNGGPSSGAPPPSGGGKCNTATCVLGRLSQELH RLQTYPRTNTGSNTY-NH2 (SEQ ID NO: 10). Compound 1A is a Polypeptide Conjugate of Compound 4A covalently attached inframe to Compound 6A through a glycine-glycine-glycine peptide linker. Compound 2 has the following sequence HGEGTFTSDLSKQLEEEAVRLFIEWLKQGGPSKEIISGGGKCNTATCVLGRLSQELHRLQ TYPRTNTGSNTY (SEQ ID NO: 11). Its C-terminally amidated form is Compound 2A, a Polypeptide Conjugate of the present invention, having the sequence HGEGTFTSDLSKQLEEEAVRLFIEWLKQGGPSKEIISGGGKCNTATCVLGRLSQELHRLQ TYPRTNTGSNTY-NH2 (SEQ ID NO: 12). Compound 2A is a Polypeptide Conjugate of Compound 5 covalently attached in-frame to Compound 6A through a glycine-glycine-glycine peptide linker.

The present study has characterized the metabolic actions and pK of Cmpd 1A and Cmpd 2A, which comprise analogs of exendin-4 covalently linked to the amylin mimetic davalintide (Cmpd 6A). As disclosed in the Examples, the effect of 4 weeks of constant subcutaneous infusion of Cmpd 1A and Cmpd 2A (at 3, 10, 30 and 100 nmol/kg/d) were compared to single and co-administration of the parent peptides Cmpd 6A, Cmpd 5 and Cmpd 4A (at 2.8, 15 and 7.2 nmol/kg/; maximum efficacious dose for weight loss) in diet-induced obese (DIO) male Sprague Dawley rats. In brief, Cmpd 1A and Cmpd 2A were more efficacious for weight loss relative to single administration of parent peptides in DIO rats, with Cmpd 1A exhibiting greater potency and efficacy for body weight loss compared to Cmpd 2A. Cmpd 1A and Cmpd 2A dose dependently reduced body weight compared to vehicle controls. Body weight loss induced by Cmpd 1A or Cmpd 2A was associated with significantly decreased percent fat mass (at 100 nmol/kg/d dose: −12.2±1.3% for Cmpd 2A and −15.5±2.2% for Cmpd 1A; both p<0.05 vs. vehicle controls). Cmpd 2A did not alter percent lean mass, however all doses of Cmpd 1A improved body composition by increasing percent lean mass relative to vehicle controls. At the highest dose tested, total food intake was significantly reduced by both Cmpd 1A and Cmpd 2A relative to single parent peptide administration. At the highest dose of Cmpd 1A food intake was suppressed beyond that of co-administration of parent compounds Cmpd 4A and Cmpd 6A. After 28 days, no change in glucose, percent hemoglobin A1c (HbA1c), insulin, total or HDL cholesterol was observed with Cmpd 2A treatment. Likewise there was no significant effect of Cmpd 1A on total or HDL cholesterol or HbA1c levels after 28 days. However, plasma insulin and glucose levels were significantly lowered by Cmpd 1A at some doses compared to vehicle controls. Plasma triglycerides were significantly reduced by Cmpd 1A and Cmpd 2A compared to vehicle after 28 days of treatment. Plasma levels of Cmpd 1A and Cmpd 2A, measured by a specific immunoassay, were detected at increasing levels corresponding to treatment dose after both 2 and 4 weeks of treatment.

Both Cmpd 1A and Cmpd 2A induced significant body weight loss in a dose-dependent manner that, at higher doses tested, exceeded weight loss induced by maximally efficacious doses of parent hormones administered as a single peptide treatment. Weight loss elicited by the highest dose of each Polypeptide Conjugate (100 nmol/kg/d) approached 30% (vehicle corrected) and was equal to (e.g., Cmpd 2A), or greater than (e.g., Cmpd 1A), weight loss achieved by co-infusion of the parent peptides. Previous reference conjugate compounds, e.g. Compound 3A and Compound 7A, were significantly less potent at reducing body weight than parent compounds despite increased efficacy. In surprising contrast, Cmpd 1A and Cmpd 2A demonstrated superior potency than Cmpd 3A and Cmpd 7A. Whereas only ˜20% weight loss was achieved with 100 nmol/kg/d of Cmpd 3A and Cmpd 7A, similar weight loss was achieved with only 10 nmol/kg/d of Cmpd 1A and Cmpd 2A. Furthermore, the lowest dose of 3 nmol/kg/d of each of Cmpd 1A or Cmpd 2A elicited significant body weight loss of ˜11-19% compared to vehicle controls over the 4 week period, similar to that of both Cmpd 6A (12%), Cmpd 4A (14%) and Cmpd 5 (17%). Cmpd 1A also was more efficacious than Cmpd 2A for weight loss (−37% vs.-28%, respectively).

Body weight loss induced by Cmpd 1A and Cmpd 2A was associated with dose dependent reductions in food intake. Additionally, Cmpd 1A treatment was associated with reduced fat mass and concomitant preservation of lean mass, an improvement in body composition. Although the control DIO rats were not hyperinsulinemic or diabetic, modest effects on reducing insulin, but not unexpectedly glucose or HbA1c, were observed with Cmpd 1A, as well as with parent peptide single or co-administration.

Plasma levels of Cmpd 1A and Cmpd 2A were measured on day 14 and day 28 of the rat study described in the Examples, using a specific assay. While approximately dose-dependent increases in both Polypeptide Conjugates were observed, levels of Cmpd 2A appeared to be much higher than those of Cmpd 1A especially after 28 days. However, when the PK profile was aligned with each compound's pharmacological (PD) profile, the PD effect of Cmpd 2A did not extend to match its PK profile. In other words, although Cmpd 2A may have been accumulating in the serum during the study period, its PD effect had waned compared to Cmpd 1A. Without intending to be bound by theory, this discrepancy may represent an early development of antibodies towards Cmpd 2A. Even if this difference was due to some interference (plasma Ab) in the Cmpd 2A PK assay, the concomitant extended PD and PK profiles of Cmpd 1A in comparison provides another important, surprisingly superior property of Cmpd 1A over Cmpd 2A. Overall, Cmpd 1A and Cmpd 2A exerted marked, fat-specific weight loss in DIO rats, superior to Cmpd 3A, with Cmpd 1A superior to both of the other compounds. Cmpd 1A treatment, displaying a very high degree of potency and efficacy, also demonstrated improvements on other metabolic parameters including triglyceride-lowering and anti-diabetic actions, as well as lack of or reduced nausea, compared to reference compounds and conjugates (see the Examples).

In one embodiment the Polypeptide Conjugates described herein include those that have been chemically derivatized. Such derivatized Polypeptide Conjugates include conjugation to one or more polymer moieties, such as polyethylene glycol (PEG) or fatty acid chains of various lengths (e.g., stearyl, palmitoyl, octanoyl, etc.), or by the addition of polyamino acids, such as poly-his, poly-arg, poly-lys, and poly-ala. Modifications can also include small molecules moieties, such as short alkyls and constrained alkyls (e.g., branched, cyclic, fused, adamantyl) and aromatic groups. The polymer moieties will typically have a molecular weight from about 500 to about 60,000 Daltons. The polymer may be linear or branched. Such derivatizations can take place at the N- or C-terminus or at a side chain of an amino acid residue, e.g. lysine epsilon amino group, aspartic acid, glutamic acid, cysteine sulfhydryl group, within the Polypeptide Conjugate. Alternatively, derivatization can occur at multiple sites throughout the conjugate polypeptide. To provide a site(s) for derivatization, substitution of one or more amino acids with, or addition of, a lysine, aspartic acid, glutamic acid or cysteine can be done. See for example U.S. Pat. Nos. 5,824,784 and 5,824,778, which are incorporated by reference herein.

In one embodiment the Polypeptide Conjugates can be conjugated to one, two, or three polymer moieties. In one embodiment, the compounds are linked to one polyethylene glycol. Pegylating the Polypeptide Conjugates can improve their aqueous solubility, increase plasma half-life, reduce immunogenicity and/or improve oral uptake. The polyethylene glycol can have a molecular weight from about 200 daltons to about 80,000 daltons; from about 5,000 daltons to about 60,000 daltons; from about 10,000 daltons to about 50,000 daltons; or from about 15,000 daltons to about 40,000 daltons. The polyethylene glycol may be linear or branched. In one embodiment the pegylated Polypeptide Conjugates comprises a lysine side chain to which is covalently attached via the lysine epsilon amino group a polyethylene glycol moiety. In a further embodiment the total molecular weight of the PEG moiety is at least about 10,000 Daltons, at least about 20,000 Daltons, at least about 40,000 Daltons or at least about 60,000 Daltons.

In one embodiment, compounds are linked to one or two polyethylene glycols, where the polyethylene glycol is further linked to a lipophilic moiety. In one embodiment, the polyethylene glycol in this case may have a molecular weight from about 200 to about 7,000 daltons or from about 500 to about 5,000 daltons. The lipophilic moiety may be an alkyl group (e.g., C₁₋₂₀ alkyl group; C₁₋₁₀ alkyl group; C₁₋₆ alkyl group; C₁₋₄ alkyl group), a fatty acid (e.g., C₄₋₂₈ fatty acid chain; C₈₋₂₄ fatty acid chain; C₁₀₋₂₀ fatty acid chain), cholesteryl, adamantyl, and the like. The alkyl group may be linear or branched, preferably linear. In one embodiment, the fatty acid is an acetylated fatty acid or an esterified fatty acid. The -(polyethylene glycol)-(lipophilic moiety) may be linked to the compound at a C-terminal amino acid residue, an N-terminal amino acid residue, an internal amino acid residue (e.g., an internal Lys amino acid residue), or a combination thereof (e.g., the compound is linked at the N-terminal and C-terminal amino acid residues). In such embodiments the derivative has superior oral uptake and bioavailability compared to underivatized Cmpd 1A or Cmpd 2A.

In one embodiment, the compounds are linked to a polyamino acid. Exemplary polyamino acids include poly-lysine, poly-aspartic acid, poly-serine, poly-glutamic acid, and the like. The polyamino acid may be in the D or L form, preferably the L form. The polyamino acids may comprise from 1 to 12 amino acid residues; from 2 to 10 amino acid residues; or from 2 to 6 amino acid residues. The derivative can provided enhanced duration of action compared to underivatized Cmpd 1A or Cmpd 2A.

In one embodiment, compounds are linked to a fatty acid. The fatty acid may be a C₄-C₂₈ fatty acid chain, a C₈-C₂₄ fatty acid chain, or a C₁₀-C₂₀ fatty acid chain. In one embodiment, the fatty acid is an acetylated fatty acid. In one embodiment, the fatty acid is an esterified fatty acid. The derivative can provided enhanced duration of action compared to underivatized Cmpd 1A or Cmpd 2A.

In one embodiment, the compounds are linked to albumin. The albumin may be a recombinant albumin, serum albumin, or recombinant serum albumin. In another embodiment, the compounds are linked to an albumin-fatty acid (i.e., an albumin linked to a fatty acid). The derivative can provided enhanced duration of action compared to underivatized Cmpd 1A or Cmpd 2A.

In one embodiment, the compounds are linked to an immunoglobulin or an immunoglobulin Fc region. The immunoglobulin may be IgG, IgE, IgA, IgD, or IgM. In one embodiment, the compounds are linked to an IgG Fc region or an IgM Fc region. The immunoglobulin Fc region is (i) the heavy chain constant region 2(C_{H}2) of an immunoglobulin; (ii) the heavy chain constant region 3(C_{H}3) of an immunoglobulin; or (iii) both the heavy chain constant regions 2(C_{H}2) and 3(C_{H}3) of an immunoglobulin. The immunoglobulin Fc region may further comprise the hinge region at the heavy chain constant region. Other embodiments for the immunoglobulin Fc region that can be linked to exendin analog peptides are described in WO 2008/082274, the disclosure of which is incorporated by reference herein. The derivative can provided enhanced duration of action compared to underivatized Cmpd 1A or Cmpd 2A.

When the Polypeptide Conjugates described herein are covalently linked to one or more polymers, such as those described herein, any linking group known in the art can be used. The linking group may comprise any chemical group(s) suitable for linking the peptide to the polymer. Alternatively, Polypeptide Conjugates can be directly attached to the polymer without any linking group. Exemplary linking groups include amino acids, maleimido groups, dicarboxylic acid groups, succinimide groups, or a combination of two or more thereof. Methods for linking peptides to one or more polymers are known in the art and described, for example, in U.S. Pat. No. 6,329,336; U.S. Pat. No. 6,423,685; U.S. Pat. No. 6,924,264; WO 2005/077072, WO 2007/022123, WO 2007/053946; WO 2008/058461; and WO 2008/082274, the disclosures of which are incorporated by reference herein.

The administered Polypeptide Conjugate may be in the form of a pro-drug. The term “prodrug” refers to a compound that is a drug precursor that, following administration, releases the drug in vivo via some chemical or physiological process, for example, proteolytic cleavage, or upon reaching an environment of a certain pH.

The Polypeptide Conjugates described herein may be prepared using biological, chemical, and/or recombinant DNA techniques that are known in the art. Exemplary methods are described in U.S. Pat. No. 6,872,700; WO 2007/139941; WO 2007/140284; WO 2008/082274; WO 2009/011544; and US Publication No. 2007/0238669, the disclosures of which are incorporated herein by reference. Other methods for preparing the Polypeptide Conjugates are set forth herein.

The Polypeptide Conjugates described herein may be prepared using standard solid-phase peptide synthesis techniques, such as an automated or semiautomated peptide synthesizer. Typically, using such techniques, an alpha-N-carbamoyl protected amino acid and an amino acid attached to the growing peptide chain on a resin are coupled at room temperature in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from Applied Biosystems Inc. (Foster City, Calif.).

For chemical synthesis solid phase peptide synthesis can be used for the Polypeptide Conjugates, since in general solid phase synthesis is a straightforward approach with excellent scalability to commercial scale, and is generally compatible with relatively long Polypeptide Conjugates. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., Introduction to Cleavage Techniques, Applied Biosystems, Inc., 1990, pp. 6-12). Peptides may be also be assembled using an Advanced Chem Tech Synthesizer (Model MPS 350, Louisville, Ky.).

Peptides may be purified by RP-HPLC (preparative and analytical) using a Waters Delta Prep 3000 system. A C4, C8 or C18 preparative column (10μ, 2.2×25 cm; Vydac, Hesperia, Calif.) may be used to isolate peptides, and purity may be determined using a C4, C8 or C18 analytical column (5μ, 0.46×25 cm; Vydac). Solvents (A=0.1% TFA/water and B=0.1% TFA/CH₃CN) may be delivered to the analytical column at a flow rate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen et al, The Pico Tag Method: A Manual of Advanced Techniques for Amino Acid Analysis, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer.

Non-peptide Polypeptide Conjugates may be prepared by art-known methods. For example, phosphate-containing amino acids and peptides containing such amino acids, may be prepared using methods known in the art, such as described in Bartlett et al, Biorg. Chem., 14:356-377 (1986).

The Polypeptide Conjugates may alternatively be produced by recombinant techniques well known in the art. See, e.g., Sambrook et al., Molecular Cloning: A Laboratory Manual, 2d ed., Cold Spring Harbor (1989). These Polypeptide Conjugates produced by recombinant technologies may be expressed from a polynucleotide. One skilled in the art will appreciate that the polynucleotides, including DNA and RNA, that encode such Polypeptide Conjugates may be obtained from the wild-type cDNA, e.g. exendin-4, amylin, taking into consideration the degeneracy of codon usage, and may further engineered as desired to incorporate the indicated substitutions. These polynucleotide sequences may incorporate codons facilitating transcription and translation of mRNA in microbial hosts. Such manufacturing sequences may readily be constructed according to the methods well known in the art. See, e.g., WO 83/04053. The polynucleotides above may also optionally encode an N-terminal methionyl residue. Non-peptide Polypeptide Conjugates useful in the present invention may be prepared by art-known methods. For example, phosphate-containing amino acids and peptides containing such amino acids may be prepared using methods known in the art. See, e.g., Bartlett and Landen, Bioorg. Chem. 14: 356-77 (1986).

A variety of expression vector/host systems may be utilized to contain and express a Polypeptide Conjugate coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Exemplary protocols for the recombinant expression of the protein are described herein.

As such, polynucleotide sequences are useful in generating new and useful viral and plasmid DNA vectors, new and useful transformed and transfected prokaryotic and eucaryotic host cells (including bacterial, yeast, and mammalian cells grown in culture), and new and useful methods for cultured growth of such host cells capable of expression of the present conjugate polypeptides. The polynucleotide sequences encoding Polypeptide Conjugates herein may be useful for gene therapy in instances where underproduction of Polypeptide Conjugates would be alleviated, or the need for increased levels of such would be met.

The present invention also provides for processes for recombinant DNA production of the present conjugate polypeptides. Provided is a process for producing the Polypeptide Conjugates from a host cell containing nucleic acids encoding said Polypeptide Conjugate comprising: (a) culturing said host cell containing polynucleotides encoding said Polypeptide Conjugate under conditions facilitating the expression of said DNA molecule; and (b) obtaining said conjugate polypeptide.

Host cells may be prokaryotic or eukaryotic and include bacteria, mammalian cells (such as Chinese Hamster Ovary (CHO) cells, monkey cells, baby hamster kidney cells, cancer cells or other cells), yeast cells, and insect cells.

Mammalian host systems for the expression of the recombinant protein also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing, which cleaves a “prepro” form of the protein, may also be important for correct insertion, folding and/or function. Different host cells, such as CHO, HeLa, MDCK, 293, WI38, and the like, have specific cellular machinery and characteristic mechanisms for such post-translational activities, and may be chosen to ensure the correct modification and processing of the introduced foreign protein.

Alternatively, a yeast system may be employed to generate the Polypeptide Conjugates of the present invention. The coding region of the Polypeptide Conjugates DNA is amplified by PCR. A DNA encoding the yeast pre-pro-alpha leader sequence is amplified from yeast genomic DNA in a PCR reaction using one primer containing nucleotides 1-20 of the alpha mating factor gene and another primer complementary to nucleotides 255-235 of this gene (Kurjan and Herskowitz, Cell, 30: 933-43 (1982)). The pre-pro-alpha leader coding sequence and Polypeptide Conjugate coding sequence fragments are ligated into a plasmid containing the yeast alcohol dehydrogenase (ADH2) promoter, such that the promoter directs expression of a fusion protein consisting of the pre-pro-alpha factor fused to the mature conjugate polypeptide. As taught by Rose and Broach, Meth. Enz. 185: 234-79, Goeddel ed., Academic Press, Inc., San Diego, Calif. (1990), the vector further includes an ADH2 transcription terminator downstream of the cloning site, the yeast “2-micron” replication origin, the yeast leu-2d gene, the yeast REP1 and REP2 genes, the E. coli beta-lactamase gene, and an E. coli origin of replication. The beta-lactamase and leu-2d genes provide for selection in bacteria and yeast, respectively. The leu-2d gene also facilitates increased copy number of the plasmid in yeast to induce higher levels of expression. The REP1 and REP2 genes encode proteins involved in regulation of the plasmid copy number.

The DNA construct described in the preceding paragraph is transformed into yeast cells using a known method, e.g., lithium acetate treatment (Steams et al., Meth. Enz. 185: 280-97 (1990)). The ADH2 promoter is induced upon exhaustion of glucose in the growth media (Price et al., Gene 55: 287 (1987)). The pre-pro-alpha sequence effects secretion of the fusion protein from the cells. Concomitantly, the yeast KEX2 protein cleaves the pre-pro sequence from the mature Polypeptide Conjugates (Bitter et al., Proc. Natl. Acad. Sci. USA 81: 5330-4 (1984)).

Polypeptide Conjugates of the invention may also be recombinantly expressed in yeast, e.g. Pichia, using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted Polypeptide Conjugate is purified from the yeast growth medium by, e.g., the methods used to purify said Polypeptide Conjugate from bacterial and mammalian cell supernatants.

Alternatively, the DNA encoding a Polypeptide Conjugate may be cloned into a baculovirus expression vector, e.g. pVL1393 (PharMingen, San Diego, Calif.). This conjugate-polypeptide-encoding vector is then used according to the manufacturer's directions (PharMingen) or known techniques to infect Spodoptera frugiperda cells, grown for example in sF9 protein-free media, and to produce recombinant protein. The protein is purified and concentrated from the media using methods known in the art, e.g. a heparin-Sepharose column (Pharmacia, Piscataway, N.J.) and sequential molecular sizing columns (Amicon, Beverly, Mass.), and resuspended in appropriate solution, e.g. PBS. SDS-PAGE analysis can be used to characterize the protein, for example by showing a single band that confirms the size of the desired conjugate protein, as can full amino acid amino acid sequence analysis, e.g. Edman sequencing on a Proton 2090 Peptide Sequencer, or confirmation of its N-terminal sequence.

For example, the DNA sequence encoding the predicted mature Polypeptide Conjugate may be cloned into a plasmid containing a desired promoter and, optionally, a leader sequence (see, e.g., Better et al., Science 240: 1041-3 (1988)). The sequence of this construct may be confirmed by automated sequencing. The plasmid is then transformed into E. coli, strain MC1061, using standard procedures employing CaCl2 incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will affect secretion of the mature Polypeptide Conjugate and be cleaved during secretion. The secreted recombinant Polypeptide Conjugate is purified from the bacterial culture media by the method described herein.

Alternatively, the Polypeptide Conjugates may be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The Polypeptide Conjugate coding sequence is cloned into a nonessential region of the virus, such as the polyhedrin gene, and placed under control of the polyhedrin promoter. Successful insertion of a Polypeptide Conjugate will render the polyhedrin gene inactive and produce recombinant virus lacking coat protein coat. The recombinant viruses are then used to infect S. frugiperda cells or Trichoplusia larvae in which Polypeptide Conjugate of the present invention is expressed (Smith et al., J. Virol. 46: 584 (1983); Engelhard et al., Proc. Natl. Acad. Sci. USA 91: 3224-7 (1994)).

In another example, the DNA sequence encoding the Polypeptide Conjugates may be amplified by PCR and cloned into an appropriate vector, for example, pGEX-3× (Pharmacia, Piscataway, N.J.). The pGEX vector is designed to produce a fusion protein comprising glutathione-S-transferase (GST), encoded by the vector, and a protein encoded by a DNA fragment inserted into the vector's cloning site. The primers for the PCR may be generated to include, for example, an appropriate cleavage site. The recombinant fusion protein may then be cleaved from the GST portion of the fusion protein. The pGEX-3×/Polypeptide Conjugate construct is transformed into E. coli XL-1 Blue cells (Stratagene, La Jolla, Calif.), and individual transformants are isolated and grown at 37 degrees C. in LB medium (supplemented with carbenicillin) to an optical density at wavelength 600 nm of 0.4, followed by further incubation for 4 hours in the presence of 0.5 mM Isopropyl beta-D-Thiogalactopyranoside (Sigma Chemical Co., St. Louis, Mo.). Plasmid DNA from individual transformants is purified and partially sequenced using an automated sequencer to confirm the presence of the desired conjugate polypeptide-encoding gene insert in the proper orientation.

The fusion protein, when expected to be produced as an insoluble inclusion body in the bacteria, may be purified as follows. Cells are harvested by centrifugation; washed in 0.15 M NaCl, 10 mM Tris, pH 8, 1 mM EDTA; and treated with 0.1 mg/mL lysozyme (Sigma Chemical Co.) for 15 min. at room temperature. The lysate is cleared by sonication, and cell debris is pelleted by centrifugation for 10 min. at 12,000×g. The fusion protein-containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg++ and Ca++. The fusion protein is further purified by fractionating the resuspended pellet in a denaturing SDS polyacrylamide gel (Sambrook et al., supra). The gel is soaked in 0.4 M KCl to visualize the protein, which is excised and electroeluted in gel-running buffer lacking SDS. If the GST/Polypeptide Conjugate fusion protein is produced in bacteria as a soluble protein, it may be purified using the GST Purification Module (Pharmacia Biotech).

The fusion protein may be subjected to digestion to cleave the GST from the mature conjugate polypeptide. The digestion reaction (20-40 μg fusion protein, 20-30 units human thrombin (4000 U/mg (Sigma) in 0.5 mL PBS) is incubated 16-48 hrs. at room temperature and loaded on a denaturing SDS-PAGE gel to fractionate the reaction products. The gel is soaked in 0.4 M KCl to visualize the protein bands. The identity of the protein band corresponding to the expected molecular weight of the Polypeptide Conjugate may be confirmed by partial amino acid sequence analysis using an automated sequencer (Applied Biosystems Model 473A, Foster City, Calif.).

In a particularly exemplary method of recombinant expression of the Polypeptide Conjugates of the present invention, 293 cells may be co-transfected with plasmids containing the Polypeptide Conjugates cDNA in the pCMV vector (5′ CMV promoter, 3′ HGH poly A sequence) and pSV2neo (containing the neo resistance gene) by the calcium phosphate method. In one embodiment, the vectors should be linearized with ScaI prior to transfection. Similarly, an alternative construct using a similar pCMV vector with the neo gene incorporated can be used. Stable cell lines are selected from single cell clones by limiting dilution in growth media containing 0.5 mg/mL G418 (neomycin-like antibiotic) for 10-14 days. Cell lines are screened for Polypeptide Conjugates expression by ELISA or Western blot, and high-expressing cell lines are expanded for large scale growth.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection, and its presence allows growth and recovery of cells that successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection for dhfr, that confers resistance to methotrexate; gpt, that confers resistance to mycophenolic acid; neo, that confers resistance to the aminoglycoside, G418; also, that confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, beta-glucuronidase and its substrate, GUS, and luciferase and its substrate, luciferin.

The Polypeptide Conjugates of the present invention may be produced using a combination of both automated peptide synthesis and recombinant techniques. For example, a Polypeptide Conjugate of the present invention may contain a combination of modifications including deletion, substitution, insertion and derivatization by pegylation (or other moiety, e.g. polymer, fatty acyl chain, C-terminal amidation). Such a Polypeptide Conjugate may be produced in stages. In the first stage, an intermediate Polypeptide Conjugate containing the modifications of deletion, substitution, insertion, and any combination thereof, may be produced by recombinant techniques as described. Then after an optional purification step as described herein, the intermediate Polypeptide Conjugate is pegylated (or subjected to other chemical derivatization, e.g. acylation, C-terminal amidation) through chemical modification with an appropriate pegylating reagent (e.g., from NeKtar Transforming Therapeutics, San Carlos, Calif.) to yield the desired Polypeptide Conjugate derivative. One skilled in the art will appreciate that the above-described procedure may be generalized to apply to a Polypeptide Conjugate containing a combination of modifications selected from deletion, substitution, insertion, derivation, and other means of modification well known in the art and contemplated by the present invention.

C-terminal amidation can be achieved by use of a glycine amino acid-C-terminally extended precursor, synthesized for example in yeast (e.g. Pichia) as alpha-factor fusion protein that will be secreted into culture medium. After purification, the C-terminal glycine of the Polypeptide Conjugate precursor will converted to amide by enzymatic amidation, e.g. peptidylglycine alpha-amidating monooxygenase (PAM). See for example Cooper et al., Biochem. Biophys. Acta, 1014:247-258 (1989). See also U.S. Pat. No. 6,319,685, which is incorporated herein by reference, which teaches methods for enzymatic amidation, including an alpha-amidating enzyme from rat being sufficiently pure in alpha-amidating enzyme to exhibit a specific activity of at least about 25 mU per mg of protein, and being sufficiently free of proteolytic impurities to be suitable for use with substrates purified from natural sources or produced by recombinant DNA techniques.

Formulations. The disclosure also provides pharmaceutical compositions comprising at least one of the Polypeptide Conjugates described herein and a pharmaceutically acceptable carrier. The Polypeptide Conjugates can be present in the pharmaceutical composition in a therapeutically effective amount and can be present in an amount to provide a minimum blood plasma level for therapeutic efficacy.

Pharmaceutical compositions containing the Polypeptide Conjugates described herein may be provided for peripheral administration, such as parenteral (e.g., subcutaneous, intravenous, intramuscular), topical, nasal, or oral administration. Suitable pharmaceutically acceptable carriers and their formulation are described in standard formulation treatises, such as Remington's Pharmaceutical Sciences by Martin; and Wang et al, Journal of Parenteral Science and Technology, Technical Report No. 10, Supp. 42:2 S (1988).

The Polypeptide Conjugates described herein can be provided in parenteral compositions for injection or infusion. They can, for example, be suspended in water; an inert oil, such as a vegetable oil (e.g., sesame, peanut, olive oil, and the like); or other pharmaceutically acceptable carrier. In one embodiment, the Polypeptide Conjugates are suspended in an aqueous carrier, for example, in an isotonic buffer solution at a pH of about 3.0 to 8.0, or about 3.0 to 5.0. The compositions may be sterilized by conventional sterilization techniques or may be sterile filtered. The compositions may contain pharmaceutically acceptable auxiliary substances as required to approximate physiological conditions, such as pH buffering agents. Useful buffers include for example, acetic acid buffers. A form of repository or “depot” slow release preparation may be used so that therapeutically effective amounts of the preparation are delivered into the bloodstream over many hours or days following subcutaneous injection, transdermal injection or other delivery method. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

The Polypeptide Conjugates can also be formulated as pharmaceutically acceptable salts (e.g., acid addition salts) and/or complexes thereof. Pharmaceutically acceptable salts are non-toxic salts at the concentration at which they are administered. Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. The acetate, trifluoroacetate or hydrochloride salt forms find particular use herein. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Carriers or excipients can also be used to facilitate administration of the Polypeptide Conjugates. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents.

If desired, solutions of the above compositions may be thickened with a thickening agent such as methyl cellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

Compositions may be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

The therapeutically effective amount of the Polypeptide Conjugates described herein to treat the diseases described herein will typically be from about 0.01 μg to about 5 mg; about 0.1 μg to about 2.5 mg; about 1 μg to about 1 mg; about 1 μg to about 50 μg; or about 1 μg to about 25 μg. Alternatively, the therapeutically effective amount of the GLP-1 receptor agonist Polypeptide Conjugates may be from about 0.001 μg to about 100 μg based on the weight of a 70 kg subject; or from about 0.01 μg to about 50 μg based on the weight of a 70 kg subject. These therapeutically effective doses may be administered once/day, twice/day, thrice/day, once/week, biweekly, or once/month, depending on the formulation. The exact dose to be administered is determined, for example, by the formulation, such as an immediate release formulation or an extended release formulation. For transdermal, nasal or oral dosage forms, the dosage may be increased from about 5-fold to about 10-fold.

The conjugated polypeptides described herein, which have an anti-hyperglycemia property with enhanced weight loss property, and pharmaceutical compositions comprising the conjugated polypeptides, are useful for treating numerous metabolic diseases and conditions as described herein, where there is benefit in controlling the level of blood glucose to reduce, prevent or treat hyperglycemia and/or in controlling weight loss or food intake to prevent, treat or control overweight or obesity. Treating or preventing hyperglycemia is central to treating or preventing prediabetes, abnormal glucose intolerance, insulin resistance and diabetes. The diabetes can be Type I diabetes, Type II diabetes or gestational diabetes. The present conjugated polypeptides are very useful when these conditions, e.g. Type II diabetes or Type I diabetes, are associated with overweight or obesity or the tendency to overweight or obesity, for example as caused by over eating, stress, weight-increasing drugs such as insulin, or a genetic abnormality, or other diseases and conditions described herein as would be known to a clinician in the field. The methods for treating diabetes with and without the presence of overweight or obesity or tendency thereto, provide administering to a subject, typically a subject, in need thereof a therapeutically effective amount of one or more of the conjugated polypeptides described herein to treat the subject. The present Polypeptide Conjugates also find use in conditions where hyperglycemia is an important factor, often in the absence of overt diabetes, and further where overweight or obesity is present or may occur, such as in steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in Subjects with congenital or HIV-Associated Lipoatrophy or “Fat Redistribution Syndrome”, and in Metabolic Syndrome (Syndrome X).

Obesity and its associated disorders including overweight are common and serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. See, e.g., Kopelman, 2000, Nature 404:635-43.

Obesity reduces life-span and carries a serious risk of the co-morbidities listed above, as well disorders such as infections, varicose veins, acanthosis nigricans, eczema, exercise intolerance, insulin resistance, hypertension hypercholesterolemia, cholelithiasis, orthopedic injury, and thromboembolic disease. See e.g., Rissanen et al, 1990, Br. Med. J., 301:835-7. Obesity is also a risk factor for the group of conditions called insulin resistance syndrome, or “Syndrome X” and metabolic syndrome. The worldwide medical cost of obesity and associated disorders is enormous.

The pathogenesis of obesity is believed to be multi-factoral. A problem is that, in obese subjects, nutrient availability and energy expenditure do not come into balance until there is excess adipose tissue. The central nervous system (CNS) controls energy balance and coordinates a variety of behavioral, autonomic and endocrine activities appropriate to the metabolic status of the animal. The mechanisms or systems that control these activities are broadly distributed across the forebrain (e.g., hypothalamus), hindbrain (e.g., brainstem), and spinal cord. Ultimately, metabolic (i.e., fuel availability) and cognitive (i.e., learned preferences) information from these systems is integrated and the decision to engage in appetitive (food seeking) and consummatory (ingestion) behaviors is either turned on (meal procurement and initiation) or turned off (meal termination). The hypothalamus is thought to be principally responsible for integrating these signals and then issuing commands to the brainstem. Brainstem nuclei that control the elements of the consummatory motor control system (e.g., muscles responsible for chewing and swallowing). As such, these CNS nuclei have literally been referred to as constituting the “final common pathway” for ingestive behavior.

Neuroanatomical and pharmacological evidence support that signals of energy and nutritional homeostasis integrate in forebrain nuclei and that the consummatory motor control system resides in brainstem nuclei, probably in regions surrounding the trigeminal motor nucleus. There are extensive reciprocal connection between the hypothalamus and brainstem. A variety of CNS-directed anti-obesity therapeutics (e.g., small molecules and peptides) focus predominantly upon forebrain substrates residing in the hypothalamus and/or upon hindbrain substrates residing in the brainstem.

Obesity remains a poorly treatable, chronic, essentially intractable metabolic disorder. Accordingly, a need exists for new therapies useful in weight reduction and/or weight maintenance in a subject. Such therapies would lead to a profound beneficial effect on the subject's health.

Diabetes and cardiovascular disease. Diabetes mellitus is recognized as a complex, chronic disease in which 60% to 70% of all case fatalities among diabetic patients are a result of cardiovascular complications. Diabetes is not only considered a coronary heart disease risk equivalent but is also identified as an independent predictor of adverse events, including recurrent myocardial infarction, congestive heart failure, and death following a cardiovascular incident. The adoption of tighter glucose control and aggressive treatment for cardiovascular risk factors would be expected to reduce the risk of coronary heart disease complications and improve overall survival among diabetic patients. Yet, diabetic patients are two to three times more likely to experience an acute myocardial infarction than non-diabetic patients, and diabetic patients live eight to thirteen years less than non-diabetic patients.

Understanding the high risk nature of diabetic/acute myocardial infarction patients, the American College of Cardiology/American Heart Association (“ACC/AHA”) clinical practice guidelines for the management of hospitalized patients with unstable angina or non-ST-elevation myocardial infarction (collectively referred to as “ACS”) recently recognized that hospitalized diabetic patients are a special population requiring aggressive management of hyperglycemia. Specifically, the guidelines state that glucose-lowering therapy for hospitalized diabetic/ACS patients should be targeted to achieve preprandial glucose less than 10 mg/dL, a maximum daily target than 180 mg/dL, and a post-discharge hemoglobin A1c less than 7%.

In a nationwide sample of elderly ACS patients, it was demonstrated that an increase in 30-day mortality in diabetic patients corresponded with the patients having higher glucose values upon admission to the hospital. See “Diabetic Coronary Artery Disease & Intervention,” Coronary Therapeutics 2002, Oak Brook, Ill., Sep. 20, 2002. There is increasing evidence that sustained hyperglycemia rather than transient elevated glucose upon hospital admission is related to serious adverse events. Although the ideal metric for hyperglycemia and vascular risk in patients is not readily known, it appears that the mean glucose value during hospitalization is most predictive of mortality. In a separate study of ACS patients form over forty hospitals in the United States, it was found that persistent hyperglycemia, as opposed to random glucose values upon admission to the hospital, was more predictive of in-hospital mortality. See Acute Coronary Syndrome Summit: A State of the Art Approach, Kansas City, Mo., Sep. 21, 2002. Compared with glucose values upon admission, a logistic regression model of glucose control over the entire hospitalization was most predictive of mortality. There was nearly a two-fold increased risk of mortality during hospitalization for each 10 mg/dL increase in glucose over 120 mg/dL. In a smaller cohort of consecutive diabetic/ACS patients, there was a graded increase in mortality at one year with increasing glucose levels upon hospital admission. In the hospital setting, the ACC/AHA guidelines suggest initiation of aggressive insulin therapy to achieve lower blood glucose during hospitalization.

Lipid regulation diseases. As known in the art, lipodystrophy is characterized by abnormal or degenerative conditions of the body's adipose tissue. Dyslipidemia is a disruption in the normal lipid component in the blood. It is believed that prolonged elevation of insulin levels can lead to dyslipidemia. Hyperlipidemia is the presence of raised or abnormal levels of lipids and/or lipoproteins in the blood. Hypothalamic amenorrhea is a condition in which menstruation stops for several months due to a problem involving the hypothalamus. It has been found that leptin replacement therapy in women with hypothalamic amenorrhea improves reproductive, thyroid, and growth hormone axes and markers of bone formation without causing adverse effects. See e.g., Oral et al., N Engl J. Med. 2004, 351: 959-962, 987-997. Fatty liver disease, e.g., nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). It is believed that leptin is one of the key regulators for inflammation and progression of fibrosis in various chronic liver diseases including NASH. See e.g., Ikejima et al., Hepatology Res. 33:151-154.

Additionally, without wishing to be bound by any theory, it is believed that relative insulin deficiency in type 2 diabetes, glucose toxicity, and increased hepatic free fatty acid burden through elevated delivery from intra-abdominal adipose tissue via the portal vein, are implicated as possible causes in fatty liver disorders. Indeed, it has been hypothesized that eating behavior is the key factor driving the metabolic syndrome of obesity with its many corollaries, including NASH. Accordingly, treatments aimed at decreasing food intake and increasing the number of small meals, as has already been demonstrated in type 2 diabetes, may effectively treat and prevent NASH. Drugs that promote insulin secretion and weight loss, and delay gastric emptying are also effective at improving glucose tolerance and thus may improve fatty liver with its attendant hyperinsulinemia. Thus, use of exendins, exendin analog agonists, exendin derivative agonists, particularly exendin-4, can be well suited as a treatment modality for this condition. Accordingly, Polypeptide Conjugates described herein can be useful in the treatment of fatty liver disorders.

Metabolic syndrome X. Metabolic Syndrome X is characterized by insulin resistance, dyslipidemia, hypertension, and visceral distribution of adipose tissue, and plays a pivotal role in the pathophysiology of type 2 diabetes. It has also been found to be strongly correlated with NASH, fibrosis, and cirrhosis of the liver. Accordingly, Polypeptide Conjugates described herein can be useful in the treatment of metabolic syndrome X.

Steroid induced diabetes. Glucocorticoids are well known to affect carbohydrate metabolism. In response to exogenous glucocorticoid administration, increased hepatic glucose production and reduced insulin secretion and insulin-stimulated glucose uptake in peripheral tissues is observed. Furthermore, glucocorticoid treatment alters the proinsulin (P1)/immunoreactive insulin (IRI) ratio, as known in the art. Typical characteristics of the hyperglycemia induced by glucocorticoids in subjects without diabetes include a minimal elevation of fasting blood glucose, exaggerated postprandial hyperglycemia, insensitivity to exogenous insulin, and non-responsiveness to metformin or sulfonylurea therapy. Accordingly, Polypeptide Conjugates described herein which include an amylin, exendin or davalintide biologically active (hormone domain) peptide component, or fragment or analog thereof, can be useful in the treatment of steroid induced diabetes.

Human Immunodeficiency Virus (HIV) Treatment-Induced Diabetes. Shortly after the introduction of human immunodeficiency virus (HIV)-1 protease inhibitors (PIs) into routine clinical use, reports linking PT use with the development of hyperglycemia began to appear. While approximately 1% to 6% of HIV-infected subjects who are treated with PIs will develop diabetes mellitus, a considerably larger proportion will develop insulin resistance and impaired glucose tolerance. Accordingly, Polypeptide Conjugates described herein which include an amylin, exendin or davalintide biologically active (hormone domain) peptide component, or fragment or analog thereof, can be useful in the treatment of HIV treatment-induced diabetes.

Latent Autoimmune Diabetes in Adults (LADA). Progressive autoimmune diabetes, also known as latent autoimmune diabetes in adults (LADA), is thought to be present in approximately 10% of patients diagnosed with type 2 diabetes. LADA patients have circulating antibodies to either islet cell cytoplasmic antigen or, more frequently, glutamic acid decarboxylase. These subjects exhibit clinical features characteristic of both type 1 and type 2 diabetes. Although insulin secretion is better preserved in the slowly progressing than in the rapidly progressing form of autoimmune diabetes, insulin secretion tends to deteriorate with time in LADA subjects. Accordingly, Polypeptide Conjugates described herein which include an amylin, exendin or davalintide biologically active (hormone domain) peptide component, or fragment or analog thereof, can be useful in the treatment of LADA.

The Polypeptide Conjugates described herein and pharmaceutical compositions comprising the Polypeptide Conjugates are useful for treating insulin resistance and stimulating insulin release. The Polypeptide Conjugates are particularly useful when such conditions are further associated with overweight or obesity or a tendency to overweight or obesity. The methods for treating insulin resistance provide administering to a subject in need thereof a therapeutically effective amount of a Polypeptide Conjugate described herein to treat insulin resistance in the subject. The methods for treating stimulating insulin release provide administering to a subject in need thereof a therapeutically effective amount of a Polypeptide Conjugate described herein to stimulate insulin release in the subject.

The Polypeptide Conjugates described herein and pharmaceutical compositions comprising the Polypeptide Conjugates are useful for treating postprandial hyperglycemia. The Polypeptide Conjugates are particularly useful when such conditions are further associated with overweight or obesity or a tendency to overweight or obesity. The methods for treating postprandial hyperglycemia provide administering to a subject in need thereof a therapeutically effective amount of a Polypeptide Conjugate described herein to treat postprandial hyperglycemia in the subject.

The Polypeptide Conjugates described herein and pharmaceutical compositions comprising the Polypeptide Conjugates are useful for lowering blood glucose levels and lowering HbA1c levels. The Polypeptide Conjugates are particularly useful when such conditions are further associated with overweight or obesity or a tendency to overweight or obesity. The Polypeptide Conjugates are particularly useful when such conditions are further associated with overweight or obesity or a tendency to overweight or obesity. The methods for lowering blood glucose levels provide administering to a subject in need thereof a therapeutically effective amount of a Polypeptide Conjugate described herein to lower blood glucose levels in the subject. In one embodiment, the blood glucose levels can be fasting blood glucose levels. The methods for lowering HbA1c levels provide administering to a subject in need thereof a therapeutically effective amount a Polypeptide Conjugate described herein to lower HbA1c levels in the subject. HbA1c levels are generally a long-term measure of a subject's blood glucose levels.

Also provided are methods for treating diabetes, for example, type I, type II or gestational diabetes, comprising administering a Polypeptide Conjugate sufficient to achieve an average or minimum circulating blood plasma level of a Polypeptide Conjugate of at least about 50 pg/ml for a period of at least about 12 hours, at least about 1 day, at least about 2 days, at least about 3 days, at least about 1 week, at least about 2 weeks, at least about 3 weeks, at least about 1 month, at least about 3 months, or at least about 6 months. In one embodiment, the methods comprise the administration of a Polypeptide Conjugate sufficient to achieve an average or minimum circulating blood plasma concentration of at least about 25 pg/ml, at least about 50 pg/ml, at least about 65 pg/ml, at least about 75 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 350 pg/ml, at least about 400 pg/ml, at least about 450 pg/ml, at least about 500 pg/ml, at least about 550 pg/ml or at least about 600 pg/ml of the Polypeptide Conjugate. In other embodiments, the average or minimum concentration of the Polypeptide Conjugate is between at least about 170 pg/ml and 600 pg/ml or between at least about 170 pg/ml and 350 pg/ml. In still other embodiments, the average or minimum plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the average or minimum plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or Cmpd 1A, or a derivative thereof. In other embodiments, the concentration of the Polypeptide Conjugate is the concentration of a Polypeptide Conjugate that results in a biological or therapeutic effect, e.g. lowering fasting glucose, reducing postprandial glucose excursion, reducing HbA1c, etc., equivalent to that observed with a given concentration of exendin-4, Cmpd 4A, davalintide or a combination of the exendin plus davalintide. In further embodiments, the subject is in need of or desirous of a reduction in body weight. In a further embodiment the subject is also in need of weight/food intake control, such as in need of a reduction in body weight, reducing appetite, increasing satiety, reducing food intake, slowing gastric emptying, lowering of triglycerides, improving body composition or any combination thereof, and further optionally with reduced incidence and/or severity of nausea.

Additional embodiments provide methods for the reduction of HbA1c, overall daily average blood glucose concentration, fasting blood glucose and/or postprandial blood glucose by administering, for example to a subject in need of a reduction in HbA1c, daily average blood glucose, or fasting glucose, an amount of a Polypeptide Conjugate sufficient to achieve an average or minimum circulating blood plasma level of an exendin, a Polypeptide Conjugate of at least about 50 pg/ml for a period of at least about 12 hours, at least about 1 day, at least about 2 days, at least about 3 days, at least about 1 week, at least about 2 weeks, at least about 3 weeks, at least about 1 month, at least about 3 months, or at least about 6 months. In one embodiment, the methods comprise the administration of a Polypeptide Conjugate sufficient to achieve an average or minimum circulating blood plasma concentration of at least about 25 pg/ml, at least about 65 pg/ml, at least about 75 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 350 pg/ml, at least about 400 pg/ml, at least about 450 pg/ml, at least about 500 pg/ml, at least about 550 pg/ml or at least about 600 pg/ml of the Polypeptide Conjugate. In other embodiments, the average or minimum concentration of the Polypeptide Conjugate is between at least about 170 pg/ml and 600 pg/ml or between at least about 170 pg/ml and 350 pg/ml. In still other embodiments, the average or minimum plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the average or minimum plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or Cmpd 1A or a derivative thereof. In other embodiments, the concentration of the Polypeptide Conjugate is the concentration of a Polypeptide Conjugate that results in a biological or therapeutic effect, e.g. lowering HbA1c, equivalent to that observed with a given concentration of exendin-4, Cmpd 4A, davalintide or a combination of the exendin plus davalintide. In one embodiment, the average or minimum circulating blood plasma concentrations are achieved for a period of about 2, about 3, about 4, about 5, about 6, or about 7 days. In a further embodiment, the average or minimum plasma concentrations are achieved for a period of about 1, about 2, about 3, about 4, about 5, about 6, about 7, about 8, about 9, about 10, about 11, about 12, about 13, about 14, about 15 or about 16 weeks. In still a further embodiment, the average or minimum plasma concentrations are achieved for a period of about 5, about 6, about 7, about 8, about 9, about 10, about 11, or about 12 months. Any method for determining circulating blood concentrations of exendin or exendin agonist or davalintide may be employed with the claimed methods. In further embodiments, the subject is in need of or desirous of a reduction in body weight. In a further embodiment the subject is also in need of weight/food intake control, such as in need of a reduction in body weight, reducing appetite, increasing satiety, reducing food intake, slowing gastric emptying, lowering of triglycerides, improving body composition or any combination thereof, and further optionally with reduced incidence and/or severity of nausea.

Additionally is provided a method for reducing the increase in postprandial blood glucose concentration compared to preprandial blood glucose concentration, such that the difference between blood glucose concentration before and after a meal is reduced. This results in a lessening of the variation in blood glucose concentrations during the day as determined, for example, by 7 point self monitored blood glucose as described herein. This method comprises administering an amount of a Polypeptide Conjugate sufficient to achieve an average or minimum circulating blood plasma level of a Polypeptide Conjugate of at least about 50 pg/ml for a period of at least about 12 hours, at least about 1 day, at least about 2 days, at least about 3 days, at least about 1 week, at least about 2 weeks, at least about 3 weeks, at least about 1 month, at least about 3 months, or at least about 6 months. In one embodiment, the methods comprise the administration of a Polypeptide Conjugate sufficient to achieve an average or minimum circulating blood plasma concentration of at least about 25 pg/ml, at least about 65 pg/ml, at least about 75 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 350 pg/ml, at least about 400 pg/ml, at least about 450 pg/ml, at least about 500 pg/ml, at least about 550 pg/ml or at least about 600 pg/ml of the Polypeptide Conjugate. In other embodiments, the average or minimum concentration of the Polypeptide Conjugate is between at least about 170 pg/ml and 600 pg/ml or between at least about 170 pg/ml and 350 pg/ml. In still other embodiments, the average or minimum plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the average or minimum plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or Cmpd 1A, or a derivative thereof. In other embodiments, the concentration of the Polypeptide Conjugate is the concentration of a Polypeptide Conjugate that results in a biological or therapeutic effect, e.g. reducing postprandial blood glucose excursions, average daily blood glucose, etc., equivalent to that observed with a given concentration of exendin-4, Cmpd 4A, davalintide or a combination of the exendin plus davalintide. In one embodiment, the average or minimum circulating blood plasma concentrations are achieved for a period of about 2, about 3, about 4, about 5, about 6, or about 7 days. In a further embodiment, the average or minimum plasma concentrations are achieved for a period of about 1, about 2, about 3, about 4, about 5, about 6, about 7, about 8, about 9, about 10, about 11, about 12, about 13, about 14, about 15 or about 16 weeks. In still a further embodiment, the average or minimum plasma concentrations are achieved for a period of about 5, about 6, about 7, about 8, about 9, about 10, about 11, or about 12 months. Any method for determining circulating blood concentrations of exendin or exendin agonist or davalintide may be employed with the claimed methods. In further embodiments, the subject is in need of or desirous of a reduction in body weight. In a further embodiment the subject is also in need of weight/food intake control, such as in need of a reduction in body weight, reducing appetite, increasing satiety, reducing food intake, slowing gastric emptying, lowering of triglycerides, improving body composition or any combination thereof, and further optionally with reduced incidence and/or severity of nausea.

The Polypeptide Conjugates described herein and pharmaceutical compositions comprising the Polypeptide Conjugates are useful for reducing gastric motility and delaying gastric emptying. The Polypeptide Conjugates are particularly useful when such conditions are further associated with overweight or obesity or a tendency to overweight or obesity. The methods for reducing gastric motility provide administering to a subject in need thereof a therapeutically effective amount of a Polypeptide Conjugate described herein to reduce gastric motility in the subject. The methods for delaying gastric emptying provide administering to a subject in need thereof a therapeutically effective amount of a Polypeptide Conjugate described herein to delay gastric emptying in the subject.

The Polypeptide Conjugates described herein and pharmaceutical compositions comprising the Polypeptide Conjugates are useful for reducing food intake, reducing appetite, increasing satiety, and reducing weight. The Polypeptide Conjugates are particularly useful when such conditions are further associated with hyperglycemia, e.g. diabetes, or a tendency to hyperglycemia. The methods for reducing food intake provide administering to a subject in need thereof a therapeutically effective amount of a Polypeptide Conjugate described herein to reduce food intake in the subject. The methods for reducing appetite provide or increasing satiety administering to a subject in need thereof a therapeutically effective amount of a Polypeptide Conjugate described herein to reduce appetite in the subject. The methods for treating reducing weight provide administering to a subject in need thereof a therapeutically effective amount of a Polypeptide Conjugate described herein to reduce weight in the subject. In the methods described herein, the subject may be in need of a reduced intake in food, of a reduced appetite, or of reduced weight. In other methods described herein, the subject may be desirous of having a reduced intake in food, of having a reduced appetite, or of having a reduced weight. The subject may be of any weight, and can be overweight or obese.

The Polypeptide Conjugates described herein and pharmaceutical compositions comprising the Polypeptide Conjugates are useful for treating overweight and obesity. The Polypeptide Conjugates are particularly useful when such conditions are further associated with hyperglycemia, e.g. diabetes, or a tendency to hyperglycemia The methods for treating overweight provide administering to a subject in need thereof a therapeutically effective amount of a Polypeptide Conjugate described herein described herein to treat overweight in the subject. The methods for treating obesity provide administering to a subject in need thereof a therapeutically effective amount of a Polypeptide Conjugate described herein to treat obesity in the subject.

The Polypeptide Conjugates described herein and pharmaceutical compositions comprising the Polypeptide Conjugates are useful for improving body composition, e.g. with an improved lean muscle to body fat ratio, typically in conjunction with reducing weight. The Polypeptide Conjugates are particularly useful when such conditions are further associated with hyperglycemia, e.g. diabetes, or a tendency to hyperglycemia. The methods for improving body composition provide administering to a subject in need thereof a therapeutically effective amount of a Polypeptide Conjugate described herein to improve body composition, and optionally reduce weight, in the subject.

In one embodiment, the present application provides methods for reducing weight in a subject desirous or in need thereof, where the method comprises the administration of an amount of a Polypeptide Conjugate effective to cause weight reduction in the subject. In another embodiment, the method comprises the chronic or sustained administration a Polypeptide Conjugate effective to cause weight reduction to the subject. In still another embodiment, the weight reduction is due to a reduction in body fat or adipose tissue without a corresponding reduction in lean body mass or muscle mass. In still another embodiment, the reduction in body weight due to loss of body fat is greater than the reduction in weight due to loss of lean body mass or muscle mass. In one embodiment the reduction in body fat as compared to lean tissue or muscle is based on an absolute weight basis while in another embodiment it is based a percent of weight lost basis. In one embodiment, the loss of visceral fat is greater than the loss of non-visceral fat. In another embodiment, the loss of non-visceral fat is greater than the loss of visceral fat. In yet another embodiment the application provides methods for altering body composition, for example by reducing the ratio of fat to lean tissue, reducing the percent body fat, or increasing the percent lean tissue in an individual.

As used herein, “weight reduction” refers to a decrease in a subject's body weight. In one embodiment, the decrease in body weight is a result of a preferential decrease in the body fat of the subject. In one embodiment, the loss of visceral fat is greater than the loss of non-visceral fat. In another embodiment, the loss of non-visceral fat is greater than the loss of visceral fat. While the invention does not depend on any particular reduction in the subject's weight, the methods described herein will, in various embodiments, reduce the subject's weight by at least about 1%, at least about 2%, at least about 3%, at least about 4%, at least about 5%, at least about 10%, at least about 15, at least about 20%, at least about 30%, at least about 40%, at least about 50%, at least about 60%, or at least about 70% compared to the subject's body weight prior to initiation of the methods disclosed herein. In various embodiments, the weight reduction occurs over a period of about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year or more. In other embodiments, the subject may lose about 5, about 6, about 7, about 8, about 9, about 10, about 15, about 20, about 25, about 30, about 35, about 40, about 45, about 50 about 100, about 125, about 150, about 175, about 200 or more pounds. A reduction in weight can be measured using any reproducible means of measurement. In one embodiment, weight reduction can be measured by calculating a subject's body mass index and comparing that subject's BMI over a period of time. Body mass index can be calculated using any method available, for example by using a nomogram or similar device.

The Polypeptide Conjugates described herein and pharmaceutical compositions comprising the Polypeptide Conjugates are useful for treating dyslipidemia. The Polypeptide Conjugates are particularly useful when such conditions are further associated with hyperglycemia, e.g. diabetes, or a tendency to hyperglycemia. The methods for treating dyslipidemia provide administering to a subject in need thereof a therapeutically effective amount of a Polypeptide Conjugate described herein described herein to treat dyslipidemia in the subject. The methods for treating dyslipidemia provide administering to a subject in need thereof a therapeutically effective amount of a Polypeptide Conjugate described herein to treat dyslipidemia in the subject.

The Polypeptide Conjugates described herein and pharmaceutical compositions comprising the Polypeptide Conjugates are useful for treating hypertriglyceridemia. The Polypeptide Conjugates are particularly useful when such conditions are further associated with hyperglycemia, e.g. diabetes, or a tendency to hyperglycemia. The methods for treating hypertriglyceridemia provide administering to a subject in need thereof a therapeutically effective amount of a Polypeptide Conjugate described herein described herein to treat hypertriglyceridemia in the subject. The methods for treating hypertriglyceridemia provide administering to a subject in need thereof a therapeutically effective amount of a Polypeptide Conjugate described herein to treat hypertriglyceridemia in the subject.

The Polypeptide Conjugates described herein and pharmaceutical compositions comprising the Polypeptide Conjugates are useful for treating steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in Subjects with congenital or HIV-Associated Lipoatrophy or “Fat Redistribution Syndrome”, and/or Metabolic Syndrome (Syndrome X). The Polypeptide Conjugates are particularly useful when such conditions are further associated with hyperglycemia, e.g. diabetes, or a tendency to hyperglycemia, or overweight or obesity or a tendency to overweight or obesity. The methods for treating steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in Subjects with congenital or HIV-Associated Lipoatrophy or “Fat Redistribution Syndrome”, and/or Metabolic Syndrome (Syndrome X) provide administering to a subject in need thereof a therapeutically effective amount of a Polypeptide Conjugate described herein described herein to treat the disease/condition in the subject. The methods for treating steroid induced diabetes, Human Immunodeficiency Virus (HIV) treatment-induced diabetes, latent autoimmune diabetes in adults (LADA), nonalcoholic steatohepatitis (NASH) and nonalcoholic fatty liver disease (NAFLD), diabetes development in Subjects with congenital or HIV-Associated Lipoatrophy or “Fat Redistribution Syndrome”, and/or Metabolic Syndrome (Syndrome X) provide administering to a subject in need thereof a therapeutically effective amount of a Polypeptide Conjugate described herein to treat the disease/condition in the subject.

In the methods described herein, the subject may be in need of the cited therapeutic effect and/or may be desirous of having the cited therapeutic effect. The subject may be of any weight, and can be overweight or obese.

The methods herein contemplate the chronic or sustained administration of an effective amount of a Polypeptide Conjugate to a subject to affect the desired results as described herein.

The methods disclosed can be used on any individual subject in need of such methods or individual subjects for whom practice of the methods is desired. These individuals may be any mammal including, but not limited to, humans, dogs, horses, cows, pigs, and other commercially valuable or companion animals.

In some embodiments, the Polypeptide Conjugate is given by chronic administration. As used herein, “chronic administration” refers to administration of the agent(s) in a continuous mode as opposed to an acute mode, so as to maintain the plasma concentration needed to obtain the desired therapeutic effect (activity) for an extended period of time. In one aspect, “chronic administration” refers to the administration of the Polypeptide Conjugate in a continuous mode, so as to maintain a plasma concentration at or above the therapeutically effective or desired amount. In one embodiment, such chronic administration maintains an average plasma the Polypeptide Conjugate concentration of at least about 25 pg/ml, at least about 50 pg/ml, at least about 65 pg/ml, at least about 75 pg/ml, at least about 85 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 300 pg/ml, at least about 350 pg/ml, at least about 400 pg/ml, at least about 450 pg/ml, at least about 500 pg/ml, at least about 550 pg/ml or at least about 600 pg/ml for an extended period of time. In other embodiments, the average concentration of the Polypeptide Conjugate is between at least about 170 pg/ml and 600 pg/ml or between at least about 170 pg/ml and 350 pg/ml. In still other embodiments, the average plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the average plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or in other embodiments is Cmpd 1A, or their derivatives. In other embodiments, the concentration of the Polypeptide Conjugate is the concentration of a Polypeptide Conjugate that results in a biological or therapeutic effect, e.g. weight reduction, glucose lowering, alteration in body composition, etc., equivalent to that observed with a given concentration of exendin-4, Cmpd 4A, davalintide or a combination of the exendin plus davalintide.

In still another embodiment, chronic administration maintains the plasma concentration, either average or minimum, of the Polypeptide Conjugate for a period of at least about 12 hours or at least about 1, at least about 2, at least about 3, at least about 4, at least about 5, at least about 6, or at least about 7 days. In another embodiment, chronic administration maintains the plasma concentration of the Polypeptide Conjugate for at least 1, at least about 2, at least about 3, or at least about 4 weeks or at least about 1, at least about 2, or at least about 3 months. In other embodiments, the Polypeptide Conjugate is administered by continuous mode. As used herein, “continuous mode” refers to the introduction of the Polypeptide Conjugate into the body, for example, the circulation, and not the means of administration. Thus chronic administration by a continuous mode can result from continuous infusion, either intravenously or subcutaneously; the use of a pump or metering system, either implanted or external, for continuous or intermittent delivery; or by the use of an extended release, slow release, sustained release or long acting formulation that is administered, for example, once daily, twice weekly, weekly, twice monthly, monthly, every other month or every third month. It should be recognized that the average or minimum plasma level need not be reached immediately upon administration of the formulation, but may take anywhere from hours to days to weeks to be reached. Once reached, the average or minimum plasma concentration is then maintained for the desired period of time to have its therapeutic effect.

As used herein in the context of weight reduction or altering body composition, a “subject in need thereof” is a subject who is overweight or obese. As used herein in the context of weight reduction or altering body composition, a “desirous” subject is a subject who wishes to reduce their body weight or alter their body composition, for example, by lessening their ratio of fat to lean tissue. In one embodiment, the subject is an obese or overweight subject. In exemplary embodiments, an “overweight subject” refers to a subject with a body mass index (BMI) greater than 25, or a BMI between 25 and 30. It should be recognized, however, that meaning of overweight is not limited to individuals with a BMI of greater than 25, but refers to any subject where weight loss is desirable or indicated for medical or cosmetic reasons. While “obesity” is generally defined as a body mass index over 30, for purposes of this disclosure, any subject, who needs or wishes to reduce body weight is included in the scope of “obese.” In one embodiment, subjects who are insulin resistant, glucose intolerant, or have any form of diabetes mellitus (e.g., type 1, 2 or gestational diabetes) can benefit from this method. In another embodiment, a subject in need thereof is obese. It should be noted, however, that the method described herein may be applied to subjects who do not have and/or have not been diagnosed with impaired glucose tolerance, insulin resistance or diabetes mellitus.

As used herein in the context of treating diabetes, reducing HbA1c, controlling postprandial blood glucose, lowering fasting glucose and reducing overall daily blood glucose concentration, a subject in need thereof may include subjects with diabetes, impaired glucose tolerance, insulin resistance, or subjects unable to auto-regulate blood glucose.

HbA1c or A1c or glycated hemoglobin or glycohemoglobin as commonly used in the art refers to glycosylated hemoglobin.

In one embodiment, methods for reducing body weight, reducing the ratio of fat to lean tissue or reducing BMI are provided wherein the method comprises chronically administering the Polypeptide Conjugate to a subject in need or desirous thereof. In one embodiment, the weight loss attributed to loss of fat or adipose tissue is greater than the weight loss due to lean tissue. In another embodiment, the percent of weight reduction due to loss of lean body mass is less than about 40%, less that about 30%, less than about 20%, less than about 10%, less than about 5%, less than about 2%, less than about 1%, or 0% of the total weight reduction. In one embodiment, the Polypeptide Conjugate is administered in an extended release, slow release, sustained release or long acting formulation. In one embodiment, the Polypeptide Conjugate is administered in a polymer-based sustained release formulation. Such polymer-based sustained release formulations are described, for example, in U.S. patent application Ser. No. 09/942,631, filed Aug. 31, 2001 (now U.S. Pat. No. 6,824,822) and related application Ser. No. 11/312,371, filed Dec. 21, 2005; U.S. Provisional Application No. 60/419,388, filed Oct. 17, 2002 and related U.S. patent application Ser. Nos. 10/688,786 and 10/688,059 filed Oct. 17, 2003; U.S. Provisional Application No. 60/757,258, filed Jan. 9, 2006; U.S. Provisional Application Ser. No. 60/563,245, filed Apr. 15, 2004 and related U.S. patent application Ser. No. 11/104,877, filed Apr. 13, 2005; and U.S. patent application Ser. No. 11/107,550, filed Apr. 15, 2005, the entireties of which are incorporated herein by reference.

In any one of the embodiments or methods disclosed herein, the circulating plasma Polypeptide Conjugate concentrations may be maintained at the average given plasma concentration or within about 10%, about 15%, about 20%, or about 25% of the average given plasma concentration. In other embodiments, the circulating plasma concentrations are maintained at the average given concentration or at about 98%, about 97%, about 96%, about 95%, about 90%, about 80%, about 70%, or about 60% of the average given concentration. Plasma concentrations of Polypeptide Conjugate can be measured using any method available to the skilled artisan.

In any one of the embodiments or methods described herein, the administration of the Polypeptide Conjugate is effective to sustain a minimum circulating plasma Polypeptide Conjugate concentration of at least about 50 pg/ml for at least about 12, about 24 or about 48 hours. In other embodiments, the methods comprise the administration of the Polypeptide Conjugate sufficient to sustain a minimum circulating plasma concentration of at least about 25 pg/ml, at least about 65 pg/ml, at least about 75 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 350 pg/ml, at least about 400 pg/ml, at least about 450 pg/ml, at least about 500 pg/ml, at least about 550 pg/ml or at least about 600 pg/ml of the Polypeptide Conjugate. In other embodiments, the minimum concentration of the Polypeptide Conjugate is between at least about 170 pg/ml and 600 pg/ml or between at least about 170 pg/ml and 350 pg/ml. In still other embodiments, the minimum plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the minimum plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or Cmpd 1A, or a derivative thereof. In other embodiments, the concentration of the Polypeptide Conjugate is the concentration of a Polypeptide Conjugate that results in a biological or therapeutic effect, e.g. weight reduction, glucose lowering, alteration in body composition, etc., equivalent to that observed with a given concentration of exendin-4, Cmpd 4A, davalintide or a combination of the exendin plus davalintide. In certain embodiments the minimum concentration of the Polypeptide Conjugate is sustained for a period of at least about 2, at least about 3, at least about 4, at least about 5, at least about 6, or at least about 7 days. In various embodiments, minimum circulating plasma concentrations are sustained for at least about 2, at least about 3, at least about 4, at least about 5, at least about 6, at least about 7, at least about 8, at least about 9, at least about 10, at least about 11, at least about 12, at least about 13, at least about 14, at least about 15 or at least about 16 weeks. In further embodiments, the minimum circulating plasma levels are sustained for at least about 5, at least about 6, at least about 7, at least about 8, at least about 9, at least about 10, at least about 11 or at least about 12 months. Plasma concentrations of the Polypeptide Conjugate can be measured using any method available to the skilled artisan.

In any one of the embodiments or methods described herein, the administration of the Polypeptide Conjugate is effective to maintain an average plasma Polypeptide Conjugate concentrations of at least about 50 pg/ml for at least about 12, at least about 24 or at least about 48 hours. In other embodiments, the methods comprise the administration of a Polypeptide Conjugate sufficient to sustain an average circulating plasma concentration of at least about 25 pg/ml, at least about 65 pg/ml, at least about 75 pg/ml, at least about 100 pg/ml, at least about 150 pg/ml, at least about 170 pg/ml, at least about 175 pg/ml, at least about 200 pg/ml, at least about 225 pg/ml, at least about 250 pg/ml, at least about 350 pg/ml, at least about 400 pg/ml, at least about 450 pg/ml, at least about 500 pg/ml, at least about 550 pg/ml or at least about 600 pg/ml. pg/ml of the Polypeptide Conjugate. In other embodiments, the average concentration of the Polypeptide Conjugate is between at least about 170 pg/ml and 600 pg/ml or between at least about 170 pg/ml and 350 pg/ml. In still other embodiments, the average plasma concentration of the Polypeptide Conjugate is greater than 40 pmoles/liter, greater than 50 pmoles/liter, greater than 60 pmoles/liter, greater than 70 pmoles/liter, greater than 80 pmoles/liter, greater than 90 pmoles/liter, greater than 100 pmoles/liter, greater than 110 pmoles/liter, greater than 120 pmoles/liter, greater than 130 pmoles/liter, greater than 140 pmoles/liter, or greater than 150 pmoles/liter. In still further embodiments, the average plasma concentration of the Polypeptide Conjugate is greater than 40 pmoles/liter but less than 150 pmoles/liter or greater than 40 pmoles/liter but less than 80 pmoles/liter. In one embodiment, the Polypeptide Conjugate is Cmpd 1A or Cmpd 2A, or Cmpd 1A, or a derivative thereof. In other embodiments, the concentration of the Polypeptide Conjugate is the concentration of a Polypeptide Conjugate that results in a biological or therapeutic effect, e.g. weight reduction, glucose lowering, alteration in body composition, etc., equivalent to that observed with a given concentration of exendin-4, Cmpd 4A, davalintide or a combination of the exendin plus davalintide. In certain embodiments the average concentration of the Polypeptide Conjugate is sustained for a period of at least about 2, at least about 3, at least about 4, at least about 5, at least about 6, or at least about 7 days. In various embodiments, average circulating plasma concentrations are sustained for at least about 2, at least about 3, at least about 4, at least about 5, at least about 6, at least about 7, at least about 8, at least about 9, at least about 10, at least about 11, at least about 12, at least about 13, at least about 14, at least about 15 or at least about 16 weeks. In further embodiments, the average circulating plasma levels are sustained for at least about 5, at least about 6, at least about 7, at least about 8, at least about 9, at least about 10, at least about 11 or at least about 12 months. Plasma concentrations of the Polypeptide Conjugate can be measured using any method available to the skilled artisan.

The Polypeptide Conjugate can be administered by any method available. In one embodiment, the Polypeptide Conjugate is administered subcutaneously. In another the Polypeptide Conjugate is administered orally, or via a pump or implant. In one embodiment, the Polypeptide Conjugate is continuously administered. In another embodiment, the Polypeptide Conjugate is administered in a slow release, extended release, sustained release or long acting formulation. In any of the preceding embodiments, the Polypeptide Conjugate can be administered once per day, every other day, three times per week, twice per week, once per week, twice a month, monthly, every other month or every three months. In addition, the length of the total time of administration of the Polypeptide Conjugate can be determined by the amount of weight reduction desired. Thus, the Polypeptide Conjugate can be administered according to the methods disclosed herein for a period sufficient to achieve a given target weight, BMI or body composition after which administration can be terminated. Alternatively following achievement of the target weight, BMI or body composition, the dose of the Polypeptide Conjugate can be decreased to a level to maintain the desired target. In addition, if after the target weight is achieved, the subject regains weight, the amount of Polypeptide Conjugate can be increased or, if previously terminated, the administration can be reinitiated.

Likewise in the area of glycemic control, the Polypeptide Conjugate can be administered according to the methods disclosed herein for a period sufficient to achieve a target HbA1c, a target fasting glucose level, a target overall daily blood glucose concentration, etc. after which the plasma concentration of the Polypeptide Conjugate may be reduced to a maintenance level or discontinued. If discontinued, the administration can be resumed later if necessary. In one embodiment, the Polypeptide Conjugate is administered according to methods disclosed herein for a period sufficient to lower or stabilize fasting glucose levels, reducing or eliminating high or higher than desired fasting glucose levels.

In some embodiments, methods disclosed herein further provide that Polypeptide Conjugate is co-administered with one or more other anti-diabetic and/or anti-obesity/appetite suppressing agents. By “co-administered” is meant administration of two or more active agents as a single composition, simultaneously administered as separate solutions, or alternatively, can be administered at different times relative to one another. Such anti-diabetic agents include, but are not limited to metformin, a sulphonylurea (SU), a thiazolidinedinone (TZD) or any combination thereof. Exemplary agents include pioglitazone, rosiglitazone, glibenclamide, gliclazide, glimepiride, glipizide, gliquidone, chlorpropamide, and tolbutamide. Additional agents include dipeptidyl peptidase-4 (DPP-IV) inhibitors such as vildagliptin or sitagliptin. The Polypeptide Conjugate can also be con-administered with insulin. Co-administration can be achieved by any suitable means or dosing regimen. Anti-obesity agents known in the art or under investigation include appetite suppressants, including phenethylamine type stimulants, phentermine (optionally with fenfluramine or dexfenfluramine), diethylpropion, phendimetrazine, benzphetamine, sibutramine; rimonabant, other cannabinoid receptor antagonists; oxyntomodulin; fluoxetine hydrochloride; Qnexa (topiramate and phentermine), bupropion and zonisamide or bupropion and naltrexone; or lipase inhibitors, similar to xenical or Cetilistat or GT 389-255.

In one embodiment, methods are provided for the decrease in the frequency and/or severity of gastrointestinal effects, including nausea or the number and/or severity of nausea events, associated with a Polypeptide Conjugate administration comprising chronically administering a Polypeptide Conjugate by any of the methods described herein. Sometimes chronic administration beginning with low or lower doses can induce a tolerance to the administered Polypeptide Conjugate such that high doses that typically elicit unacceptable frequency and/or severity of gastrointestinal effects can be administered to the subject with reduced or absent gastrointestinal effects. Thus, it is contemplated that chronic administration can be initiated with suboptimal dosing of the Polypeptide Conjugate using, for example, a formulation that releases the administered Polypeptide Conjugate over a period of time where the formulation is administered weekly. Over a period of weeks, the plasma levels of the Polypeptide Conjugate will increase and eventually achieve a plateau concentration. In some embodiments, this plateau is at a concentration that could not be tolerated due to adverse gastrointestinal effects if administered in a single or initiating dose. Any suitable extended-release formulation and administration regimen can be used to achieve the plateau effect.

Accordingly, in one embodiment multiple sustained release doses are provided such that each successive dose increases the concentration of the agent or agents in the subject, wherein a therapeutically effective concentration of agent or agents is achieved in the subject. In one further embodiment each successive sustained release dose is administered such that its sustained phase overlaps with the sustained phase of the previous dose.

The disclosure also provides drug delivery devices having at least one therapeutically effective dose of the Polypeptide Conjugates described herein or the pharmaceutical composition containing the Polypeptide Conjugates described herein. The drug delivery devices can be single or multiple-use vials, single or multiple-use pharmaceutical pens, single or multiple-use cartridges, and the like. In one embodiment, the drug delivery devices contain the Polypeptide Conjugates or pharmaceutical compositions described herein in amounts capable of providing a subject with from about 7 to about 40 doses or enough doses to last about one week or about one month.

The disclosure also provides a kit comprising a container comprising a Polypeptide Conjugate as described herein, optionally with instructions for use of the Polypeptide Conjugate by the subject. The container can be a vial, cartridge, pen or other delivery device, single use or multi-use, as described herein,

Also specifically contemplated are the use of a Polypeptide Conjugate for the manufacture of a medicament for use in any of the methods of treatment described herein.

Additional embodiments include the following embodiments:
1. A Polypeptide Conjugate comprising Compound 1A or Compound 2A.
2. The Polypeptide Conjugate of embodiment 1 comprising Compound 1A.
3. The Polypeptide Conjugate of embodiment 1 comprising Compound 2A.
4. The Polypeptide Conjugate of any one of embodiments 1 to 3 wherein Compound 1A or Compound 2A is covalently linked to at least one polyethylene glycol moiety.
5. The Polypeptide Conjugate of embodiment 4 wherein the polyethylene glycol is linked to a lipophilic moiety.
6. The Polypeptide Conjugate of embodiment 5 the lipophilic moiety is an alkyl group, a fatty acid, a cholesteryl, or an adamantyl.
7. The Polypeptide Conjugate compound of any one of embodiments 1 to 3 wherein Compound 1A or Compound 2A is covalently linked to at least one fatty acid.
8. The Polypeptide Conjugate compound of any one of embodiments 1 to 3 wherein Compound 1A or Compound 2A is covalently linked to albumin.
9. The Polypeptide Conjugate of embodiment 8 wherein the albumin is linked to a fatty acid.
10. The Polypeptide Conjugate compound of any one of embodiments 1 to 3 wherein Compound 1A or Compound 2A is covalently linked to a polyamino acid.
11. The Polypeptide Conjugate of any one of embodiments 1 to 10 having an EC50 of less than 1 micromolar in a GLP-1 receptor function assay.
12. The Polypeptide Conjugate of embodiment 11 having an EC50 of less than 100 nanomolar in a GLP-1 receptor function assay.
13. The Polypeptide Conjugate of embodiment 12 having an EC50 of less than 10 nanomolar in a GLP-1 receptor function assay.
14. The Polypeptide Conjugate of embodiment 13 having an EC50 of less than 1 nanomolar in a GLP-1 receptor function assay.
15. The Polypeptide Conjugate of any one of embodiments 1 to 14 wherein the Polypeptide Conjugate has an EC50 of less than 1 micromolar in a calcitonin C1a receptor function assay.
16. The Polypeptide Conjugate of embodiment 15 wherein the Polypeptide Conjugate has an EC50 of less than 100 nanomolar in a calcitonin C1a receptor function assay.
17. The Polypeptide Conjugate of embodiment 16 wherein the Polypeptide Conjugate has an EC50 of less than 10 nanomolar in a calcitonin C1a receptor function assay.
18. The Polypeptide Conjugate of embodiment 17 wherein the Polypeptide Conjugate has an EC50 of less than 5 nanomolar in a calcitonin C1a receptor function assay.
19. The Polypeptide Conjugate of any one of embodiments 1 to 18 wherein the Polypeptide Conjugate reduces body weight more potently than exendin-4, Cmpd 4A or davalintide.
20. The Polypeptide Conjugate of any one of embodiments 1 to 19 wherein the Polypeptide Conjugate reduces body weight more potently than both exendin-4 and davalintide.
21. The Polypeptide Conjugate of any one of embodiments 1 to 20 wherein the Polypeptide Conjugate reduces body weight more efficaciously than exendin-4.
22. The Polypeptide Conjugate of any one of embodiments 1 to 20 wherein the Polypeptide Conjugate reduces body weight more efficaciously than davalintide.
23. The Polypeptide Conjugate of any one of embodiments 1 to 22 wherein the Polypeptide Conjugate reduces body weight more efficaciously than both exendin-4 and davalintide.
24. The Polypeptide Conjugate of any one of embodiments 1 to 20 wherein the Polypeptide Conjugate reduces body weight more efficaciously than co-administered maximally efficacious doses of exendin-4 and davalintide.
25. The Polypeptide Conjugate of any one of embodiments 1 to 24 wherein the Polypeptide Conjugate reduces body weight more potently and more efficaciously than exendin-4.
26. The Polypeptide Conjugate of any one of embodiments 1 to 24 wherein the Polypeptide Conjugate reduces body weight more potently and/or more efficaciously than Cmpd 3A.
27. The Polypeptide Conjugate of any one of embodiments 1 to 26 wherein the reduction in body weight occurs over a period of 4 weeks.
28. The Polypeptide Conjugate of any one of embodiments 1 to 27 wherein the reduction in body weight occurs over a period of 6 months.
29. The Polypeptide Conjugate of any one of embodiments 1 to 28 wherein the reduction in body weight occurs over a period of 1 year.
30. The Polypeptide Conjugate of any one embodiments 1 to 29 wherein the Polypeptide Conjugate has reduced kaolin intake in rats compared to exendin-4.
31. The Polypeptide Conjugate of any one of embodiments 1 to 30 wherein the Polypeptide Conjugate has reduced kaolin intake in rats compared to davalintide.
32. The Polypeptide Conjugate of any one of embodiments 1 to 31 wherein the Polypeptide Conjugate has reduced kaolin intake in rats compared to Cmpd 7A.
33. The Polypeptide Conjugate of any one of embodiments 1 to 32 wherein the Polypeptide Conjugate has reduced nausea compared to exendin-4.
34. The Polypeptide Conjugate of any one of embodiments 1 to 33 wherein the Polypeptide Conjugate has reduced nausea compared to davalintide.
35. The Polypeptide Conjugate of any one of embodiments 1 to 34 wherein the Polypeptide Conjugate has reduced nausea compared to Cmpd 7A.
36. The Polypeptide Conjugate of any one of embodiments 1 to 35 wherein the reduced nausea is a lesser severity of nausea or less frequent number of adverse events per year of nausea or both, wherein the nausea events can be mild, moderate or severe or the combined total number of nausea events.
37. The Polypeptide Conjugate of any one of embodiments 1 to 36 wherein the Polypeptide Conjugate lowers fasting plasma glucose.
38. The Polypeptide Conjugate of any one of embodiments 1 to 37 wherein the Polypeptide Conjugate increases tolerance to oral glucose load.
39. The Polypeptide Conjugate of any one of embodiments 1 to 38 wherein the Polypeptide Conjugate increases glucose-induced insulin secretion.
40. The Polypeptide Conjugate of any one of the embodiments 1 to 39 wherein the Polypeptide Conjugate delays gastric emptying for at least two, at least four or at least eight hours.
41. The Polypeptide Conjugate of any one of the embodiments 1 to 40 wherein the Polypeptide Conjugate delays gastric emptying to a greater extent than Cmpd 3A at identical doses.
42. The Polypeptide Conjugate of any one of the embodiments 1 to 41 wherein the Polypeptide Conjugate lowers plasma triglycerides to a greater extent than exendin-4, davalintide or Cmpd 3A at identical doses.
43. The Polypeptide Conjugate of any one of the embodiments 1 to 42 wherein the Polypeptide Conjugate lowers plasma triglycerides to a greater extent than Compound 2A at identical doses.
44. The compound of any one of Claims 1-43 in the form of a pharmaceutically acceptable salt.
45. A pharmaceutical composition comprising a Polypeptide Conjugate according to any one of embodiments 1 to 44 and a pharmaceutically acceptable carrier.
46. A method for treating diabetes in a subject in need thereof or desirous thereof comprising administering a therapeutically effective amount of the Polypeptide Conjugate or pharmaceutical composition of any one of embodiments 1-45 to treat diabetes in the subject.
47. The method of Claim 46, wherein the diabetes is Type 1 diabetes.
48. The method of Claim 46, wherein the diabetes is Type 2 diabetes.
49. The method of Claim 46, wherein the diabetes is gestational diabetes.
50. The method of any one of embodiments 46 to 49 wherein the subject is overweight, obese or has a tendency to overweight of obese.
51. A method for treating insulin resistance in a subject in need thereof or desirous thereof comprising administering a therapeutically effective amount of the Polypeptide Conjugate or pharmaceutical composition of any one of Claims 1-45 to treat insulin resistance in the subject.
52. A method for treating postprandial hyperglycemia in a subject in need thereof or desirous thereof comprising administering a therapeutically effective amount of the Polypeptide Conjugate or pharmaceutical composition of any one of Claims 1-45 to treat postprandial hyperglycemia in the subject.
53. A method for lowering blood glucose levels in a subject in need thereof or desirous thereof comprising administering a therapeutically effective amount of the Polypeptide Conjugate or pharmaceutical composition of any one of Claims 1-45 to lower blood glucose levels in the subject.
54. A method for lowering HbA1c levels in a subject in need thereof comprising administering a therapeutically effective amount of the Polypeptide Conjugate or pharmaceutical composition of any one of Claims 1-45 to lower HbA1c levels in the subject.
55. A method for stimulating insulin release in a subject in need thereof comprising administering a therapeutically effective amount of the Polypeptide Conjugate or pharmaceutical composition of any one of Claims 1-45 to stimulate insulin release in the subject.
56. A method for reducing gastric motility in a subject in need thereof comprising administering a therapeutically effective amount of the Polypeptide Conjugate or pharmaceutical composition of any one of Claims 1-45 to reduce gastric motility in the subject.
57. A method for delaying gastric emptying in a subject in need thereof comprising administering a therapeutically effective amount of the Polypeptide Conjugate or pharmaceutical composition of any one of Claims 1-45 to delay gastric emptying in the subject.
58. A method for reducing food intake in a subject in need or desirous thereof comprising administering a therapeutically effective amount of the Polypeptide Conjugate or pharmaceutical composition of any one of Claims 1-45 to reduce food intake in the subject.
59. A method for reducing appetite in a subject in need or desirous thereof comprising administering a therapeutically effective amount of the Polypeptide Conjugate or pharmaceutical composition of any one of Claims 1-45 to reduce appetite in the subject.
60. A method for reducing weight in a subject in need or desirous thereof comprising administering a therapeutically effective amount of the Polypeptide Conjugate or pharmaceutical composition of any one of Claims 1-45 to reduce weight in the subject.
61. A method for treating overweight in a subject in need thereof or desirous thereof comprising administering a therapeutically effective amount of the Polypeptide Conjugate or pharmaceutical composition of any one of Claims 1-45 to treat overweight in the subject.
62. A method for treating obesity in a subject in need thereof or desirous thereof comprising administering a therapeutically effective amount of the Polypeptide Conjugate or pharmaceutical composition of any one of Claims 1-45 to treat obesity in the subject.
63. The method of any one of Claims 46-62, wherein the therapeutically effective amount of the compound is from about 0.1 μg to about 5 mg.
64. The method of any one of Claims 46-62, wherein the therapeutically effective amount of the compound is from about 1 μg to about 2.5 mg.
65. The method of any one of Claims 46-62, wherein the therapeutically effective amount of the compound is from about 1 μg to about 1 mg.
66. The method of any one of Claims 46-62, wherein the therapeutically effective amount of the compound is from about 1 μg to about 50 μg.
67. The method of any one of Claims 46-62, wherein the therapeutically effective amount of the compound is from about 1 μg to about 25 μg.
68. The method of any one of Claims 46-62, wherein the therapeutically effective amount of the compound is from about 0.01 μg to about 100 μg based on the weight of a 70 kg subject.
69. The method of any one of Claims 46-62, wherein the therapeutically effective amount of the compound is from about 0.01 μg to about 50 μg based on the weight of a 70 kg subject.
70. A drug delivery device comprising at least one therapeutically effective dose of the compound or pharmaceutical composition of any one of Claims 1-45.
71. The drug delivery device of Claim 70, wherein the drug delivery device is a vial, a pharmaceutical pen, or a cartridge.
72. The drug delivery device of Claim 70 or 71, comprising about a one month supply of therapeutically effective doses.
73. A process of making the compound of any one of embodiments 1 to 45.
74. The process of embodiment 73 that comprises a recombinant process.
75. A kit comprising the Polypeptide Conjugate or pharmaceutical composition of any one of embodiments 1 to 45, optionally having instructions for use of the Polypeptide Conjugate or composition by the subject.
76. A method of any one of the above embodiments wherein administration of the Polypeptide Conjugate results in improved patient compliance compared to Compound 6A, improved reduction in severe flushing compared to Compound 6A, and/or reduced nausea compared to Compound 6A.
77. The method of embodiment 63 wherein the dose is about 0.1 to 1.0 mg per day.
78. The method of embodiment 67 wherein the dose is about 0.3 to 0.6 mg per day.
79. The method of any one of the above embodiments wherein the dose is split to twice a day (BID) or given once a day (QD).

### EXAMPLES

The following examples are for purposes of illustration only and are not intended to limit the scope of the claims.

### Example 1

### Methods for In Vivo Studies in DIO Rats

The present study characterized the metabolic actions of Cmpd 1A and Cmpd 2A. As disclosed in the Examples, the effect of 4 weeks of constant subcutaneous infusion of Cmpd 1A and Cmpd 2A (at 3, 10, 30 and 100 nmol/kg/d) were compared to single and co-administration of the parent peptides, Cmpd 6A, Cmpd 5 and Cmpd 4A (at 2.8, 15 and 7.2 nmol/kg/; maximum efficacious dose for weight loss) in diet-induced obese (DIO) male Sprague Dawley rats. Various metabolic and PK parameters were evaluated.

Animals. Male Sprague Dawley rats (CRL:CD rats, Charles River Laboratories, Wilmington, Mass.) were individually housed and maintained on high fat diet (32% kcal from fat; D12266B Research Diets, Brunswick, N.J.) for approximately 8 weeks prior to the study. At the start of testing (day 0) average body weight of the rats was 545±3.8 g.

Compounds. Compounds used in the examples include the following:

Compound 4A, a full-length C-terminally amidated exendin-4 peptide analog with a single nucleotide difference at position 14 compared to native exendin-4.
Compound 5, a chimera of the first 32 amino acids of exendin-4 having amino acid substitutions at positions 14 and 28 followed by a 5 amino acid sequence from the C-terminal of a non-mammalian (frog) GLP1;
Compound 6A, davalintide, a C-terminally amidated amylin mimetic;
Compound 2A, a Polypeptide Conjugate of Compound 5 covalently attached in-frame to
Compound 6A through an glycine-glycine-glycine peptide linker; and
Compound 1A, a Polypeptide Conjugate of Compound 4A covalently attached inframe to Compound 6A through a glycine-glycine-glycine peptide linker.

Study Design. The Polypeptide Conjugates were tested relative to vehicle (50% DMSO in sterile water) and parent compounds alone and in combination (see Table 1). The doses of Cmpd 5, Cmpd 4A and Cmpd 6A are maximally efficacious for weight loss in this model (data not shown). On day 1 rats were surgically implanted with two osmotic mini-pumps (Alzet, Durect Corporation, Cupertino, Calif.) that delivered either vehicle, Cmpd 2A, Cmpd 1A, Cmpd 5, Cmpd 4A or Cmpd 6A at a constant rate (nanomoles per kilogram of rat per day) for 4 weeks. See Table 1.

TABLE 1
Group Allocation and Treatments

| | |
|---|---|
| Group # | Treatment (nmol/kg/day) |

| | | |
|---|---|---|
| 1 | Vehicle | Vehicle |
| 2 | Vehicle | Cmpd 2A (3) |
| 3 | Vehicle | Cmpd 2A (10) |
| 4 | Vehicle | Cmpd 2A (30) |
| 5 | Vehicle | Cmpd 2A (100) |
| 6 | Vehicle | Cmpd 1A (3) |
| 7 | Vehicle | Cmpd 1A (10) |
| 8 | Vehicle | Cmpd 1A (30) |
| 9 | Vehicle | Cmpd 1A (100) |
| 10 | Vehicle | Cmpd 5 (15) |
| 11 | Vehicle | Cmpd 4A (7.2) |
| 12 | Vehicle | Cmpd 6A (2.8) |
| 13 | Cmpd 5 (15) | Cmpd 6A (2.8) |
| 14 | Cmpd 4A (7.2) | Cmpd 6A (2.8) |

Food intake and body weight were measured weekly. Body composition was assessed on day −1 and day 28 using an NMR instrument (Echo Medical Systems, Houston, Tex.). Adiposity (percent fat mass) was defined as the amount of fat mass relative to body weight (fat mass/body weight×100). Blood was collected via tail vein on day 14. On day 28 a sample of blood was drawn via the jugular vein and animals were euthanized by isoflurane overdose. Mini-pumps were immediately removed, animals were subjected to a brief NMR scan, and tissues were collected for future histological examination and preliminary toxicological assessment.

Statistical Analysis. Data were analyzed using one-way analysis of variance (ANOVA) with Newman-Keuls post-hoc comparisons. Significance was assumed for p<0.05. Graphs were generated using Prism 4 for Windows (Graphpad Software, San Diego, Calif.). All data points are expressed as mean±SEM. For the highest doses of Cmpd 1A and Cmpd 2A, several of the animals had minimal food intake and this data was included in the analysis.

Hormone and metabolite analyses. Plasma levels of study drug were measured at day 14 and termination by an ELISA. Whole blood percent hemoglobin A1c (% HbA1c), plasma triglyceride, total cholesterol, HDL cholesterol and plasma glucose at day 14 and at termination were measured using an Olympus bioanalyzer (Olympus America Diagnostics). Plasma insulin levels at day 14 and at termination were analyzed by an ELISA kit (Rat/Mouse Insulin ELISA, Linco Diagnostics).

### Example 2

### Compound 1A Provides Superior Body Weight Loss

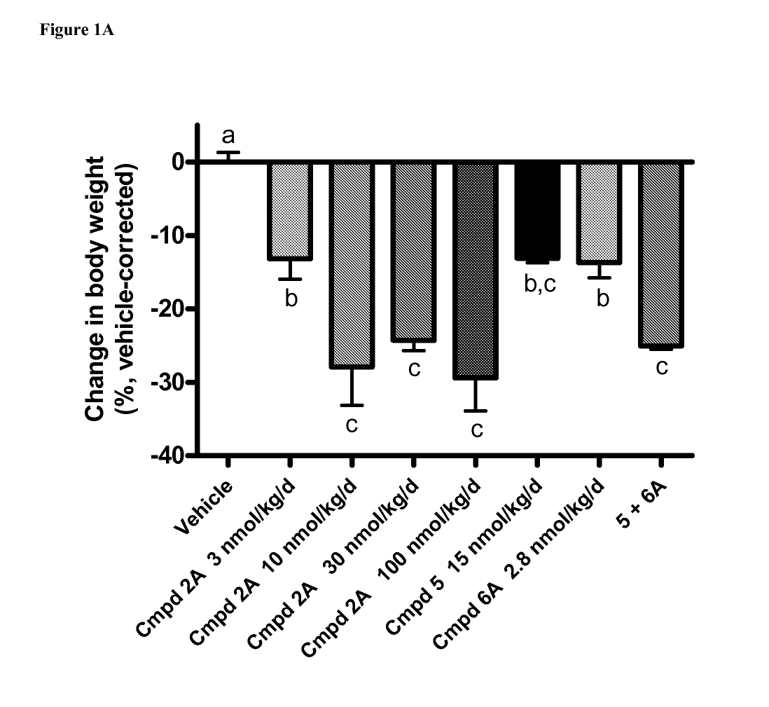

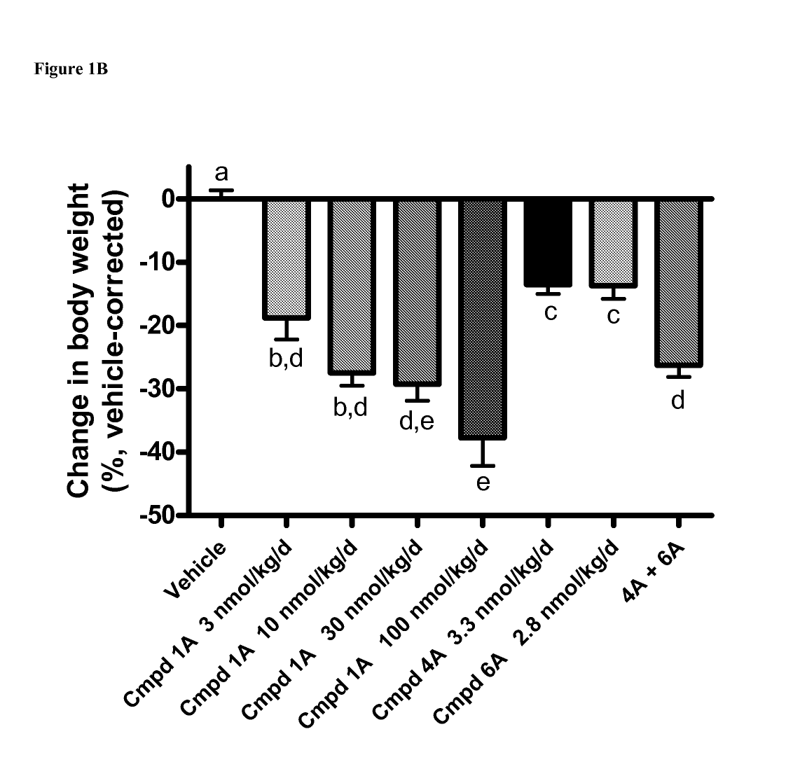

Following the method of Example 1, each Polypeptide Conjugate (Cmpd 1A and Cmpd 2A) dose-dependently reduced body weight significantly (p<0.05 relative to vehicle) at 3, 10, 30 and 100 nmol/kg/d over the 28 day treatment (see FIGS. 1A and 1B). Co-administration of the parent compounds (Cmpd 5+Cmpd 6A and Cmpd 4A+Cmpd 6A) significantly reduced body weight compared to vehicle controls and compared to Cmpd 6A alone (p<0.05). See FIGS. 1A and 1B.

As indicated in FIG. 1A, four week vehicle-corrected weight loss was: −31.5±2.7% for Cmpd 2A at 100 nmol/kg/d, −16.6±3.6% for Cmpd 5, −12.3±1.3% for Cmpd 6A, −24.3±1.3% for Cmpd 5+Cmpd 6A, p<0.05 for Cmpd 2A vs. Cmpd 6A, but not different from Cmpd 5 or co-administration of Cmpd 5+Cmpd 6A. For Cmpd 2A there was no dose-effect beyond 10 nmol/kg/d; further weight loss was not observed with doses of 30 or 100 nmol/kg/d. Maximal weight loss was achieved in the 100 nmol/kg/d group, at −27.8±5.3%. See FIG. 1A. Cmpd 2A was also several fold more potent at reducing body weight via sustained infusion than Cmpd 3A.

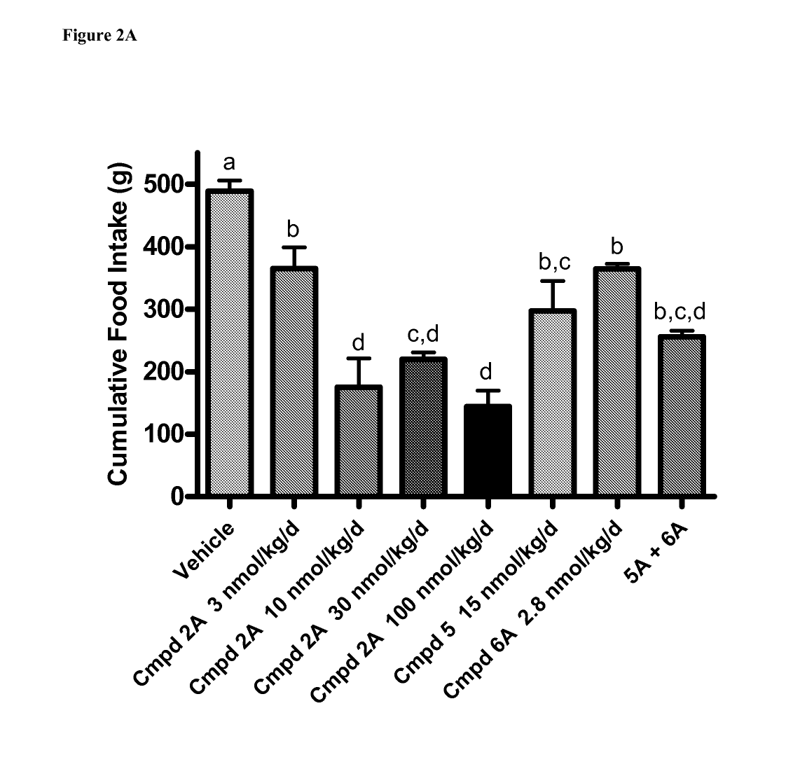

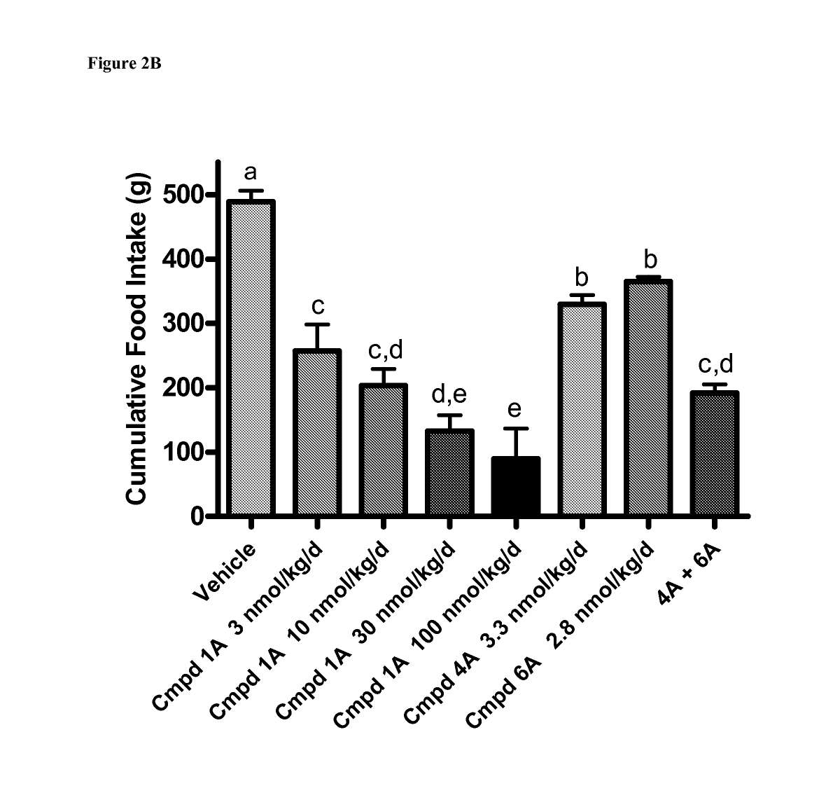

As indicated in FIG. 1B, four week vehicle-corrected weight loss was: −37.3±4.8% for Cmpd 1A at 100 nmol/kg/d, −13.5±1.5% for Cmpd 4A and −25.8±1.5% for Cmpd 4A+Cmpd 6A; p<0.05 for Cmpd 1A vs. both parent peptides alone and in combination. For Cmpd 1A dose-dependent weight loss was observed up to the highest dose tested [100 nmol/kg/d group at −37.3±4.8%]. Weight loss with Cmpd 1A at 100 nmol/kg/d was also significantly greater than weight loss observed with co-administration of maximally efficacious doses of parent compounds. See FIG. 2A. Although both Polypeptide Conjugates elicited profound weight loss over the 28 day treatment period, for Cmpd 1A weight loss at even the lowest dose tested was significantly greater than via single administration of either parent peptide. Cmpd 1A was also remarkably more potent and more efficacious than Cmpd 2A. Cmpd 1A was also approximately 10-fold more potent at reducing body weight via sustained infusion than Cmpd 3A.

### Example 3

### Compound 1A Provides a Superior Decrease in Food Intake

Following the method of Example 1, as with body weight each Polypeptide Conjugate decreased food intake significantly (p<0.05) at each dose tested relative to vehicle controls over the 28 day treatment period. See FIGS. 2A and 2B. At the highest dose tested, total food intake was significantly reduced by Cmpd 1A and by Cmpd 2A relative to single parent peptide administration, and at the highest dose of Cmpd 1A food intake was suppressed beyond that of co-administration of Cmpd 4A+Cmpd 6A.

Cumulative food intake for Cmpd 2A at 3 nmol/kg/d was significantly higher than the other doses (p<0.05). When compared to parent compound monotherapy the 10 and 100 nmol/kg/day doses were significantly lower than either parent, however the 30 nmol/kg/d dose was only significantly different from parent compound Cmpd 6A. Co-administered Cmpd 5+Cmpd 6A treatment was not significantly different from any of the Polypeptide Conjugate doses. See FIG. 2A. Cmpd 2A was more potent at reducing food intake acutely than Cmpd 3A.

All doses of Cmpd 1A decreased food intake significantly when compared to vehicle and to either parent compounds administered alone. See FIG. 2B. The highest dose of Cmpd 1A was the only dose significantly different from the combination therapy (co-administration) of the two parent compounds Cmpd 4A and Cmpd 6A. See FIG. 2B. Accordingly, Cmpd 1A provided a superior decrease in food intake. Cmpd 1A was also more potent at reducing food intake acutely than Cmpd 3A. Overall, Cmpd 1A and Cmpd 2A were more efficacious for weight loss relative to single administration of parent peptides in DIO rats, with Cmpd 1A exhibiting greater potency and efficacy for body weight loss compared to Cmpd 2A.

### Example 4

### Compound 1A Provides Improvement in Body Composition

Following the method of Example 1, at the start of the study no significant differences in baseline fat mass, expressed as either raw weight or as percent fat mass (adiposity), or in baseline lean mass were observed among the test subjects (See Table 2 and Table 3). At termination it was observed that increasing doses of Polypeptide Conjugate resulted in reductions in fat mass. Polypeptide Conjugate-induced body weight loss was associated with significantly decreased percent fat mass (at 100 nmol/kg/d dose: −12.2±1.3% for Cmpd 2A and −15.5±2.2% for Cmpd 1A; both p<0.05 vs. vehicle controls). Cmpd 2A did not alter percent lean mass, however all doses of Cmpd 1A increased percent lean mass relative to vehicle controls. Cmpd 1A and Cmpd 2A at 100 nmol/kg/d reduced fat mass significantly compared to vehicle and to administration of either parent compound groups (see Table 2 and Table 3). Cmpd 2A at 10 and 100 nmol/kg/d and Cmpd 5+Cmpd 6A co-administration treatment significantly reduced terminal lean mass relative to vehicle (see Table 2). Cmpd 1A treatment at 10, 30 and 100 nmol/kg/d, and Cmpd 4A+Cmpd 6A treatment, significantly lowered lean mass compared to vehicle controls (Table 3).

TABLE 2
Cmpd 2A baseline and terminal body composition

| | | | | Baseline | | |
|---|---|---|---|---|---|---|
| | Baseline | Baseline | Baseline | Percent | Terminal | Terminal |
| Treatment | Fat Mass | Adiposity | Lean Mass | Lean | Fat Mass | Lean Mass |
| (nmol/kg/d)¹ | (g) | (%) | (g) | Mass (%) | (g) | (g) |
| Vehicle | 90.3 ± 10.2 | 16.9 ± 1.8 | 367.5 ± 12.9 | 69.1 ± 2.2 | 93.4 ± 8.8^{a} | 356.0 ± 8.7^{a} |
| Cmpd 2A | 102.8 ± 12.9 | 19.1 ± 1.8 | 352.4 ± 5.2 | 66.4 ± 1.7 | 71.4 ± 12.9^{a,b} | 330.5 ± 7.3^{a,b} |
| (3) | | | | | | |
| Cmpd 2A | 105.1 ± 6.9 | 19.7 ± 1.0 | 351.3 ± 5.4 | 65.9 ± 1.3 | 35.8 ± 7.7^{c,d} | 280.6 ± 22.6^{b} |
| (10) | | | | | | |
| Cmpd 2A | 93.2 ± 6.4 | 17.3 ± 1.1 | 371.0 ± 5.1 | 69.0 ± 0.9 | 32.4 ± 3.8^{c,d} | 306.5 ± 13.2^{a,b} |
| (30) | | | | | | |
| Cmpd 2A | 84.1 ± 9.8 | 15.6 ± 1.5 | 379.9 ± 12.0 | 71.3 ± 1.9 | 12.7 ± 4.3^{d} | 277.8 ± 17.8^{b} |
| (100) | | | | | | |
| Cmpd 5 | 97.6 ± 9.2 | 18.4 ± 1.6 | 357.2 ± 9.4 | 67.5 ± 1.8 | 51.9 ± 8.1^{b,c} | 330.9 ± 11.3^{a,b} |
| (15) | | | | | | |
| Cmpd 6A | 86.2 ± 8.8 | 16.1 ± 1.4 | 362.1 ± 15.2 | 68.1 ± 1.6 | 54.8 ± 9.3^{b,c} | 324.6 ± 14.7^{a,b} |
| (2.8) | | | | | | |
| Cmpd 5 | 86.1 ± 4.2 | 16.1 ± 0.5 | 371.7 ± 10.4 | 69.7 ± 1.0 | 31.9 ± 1.7^{c,d} | 287.5 ± 10.0^{b} |
| (15) + Cmpd | | | | | | |
| 6A (2.8) | | | | | | |

¹Groups not sharing a superscript are significantly different from each other (p < 0.05).

TABLE 3
Cmpd 1A baseline and terminal body composition

| | | | | Baseline | | |
|---|---|---|---|---|---|---|
| | Baseline | Baseline | Baseline | Percent | Terminal | Terminal |
| Treatment | Fat Mass | Adiposity | Lean Mass | Lean | Fat Mass | Lean Mass |
| (nmol/kg/d)¹ | (g) | (%) | (g) | Mass (%) | (g) | (g) |
| Vehicle | 90.3 ± 10.2 | 16.9 ± 1.8 | 367.5 ± 12.9 | 69.1 ± 2.2 | 93.4 ± 8.8^{a} | 356 ± 8.7^{a} |
| Cmpd 1A | 98.4 ± 736 | 18.5 ± 1.2 | 351.7 ± 8.3 | 66.3 ± 1.3 | 46.6 ± 8.3^{b,c,d} | 310.1 ± 16.0^{a,b} |
| (3) | | | | | | |
| Cmpd 1A | 86.6 ± 12.2 | 15.9 ± 1.8 | 369.6 ± 10.0 | 69.2 ± 1.7 | 28.3 ± 6.5^{c,d} | 302.5 ± 14.2^{b,c} |
| (10) | | | | | | |
| Cmpd 1A | 100.2 ± 3.1 | 18.8 ± 0.4 | 350.9 ± 8.4 | 65.9 ± 0.8 | 24.1 ± 5.8^{d} | 274.7 ± 11.8^{b,c} |
| (30) | | | | | | |
| Cmpd 1A | 110.1 ± 11.3 | 20.5 ± 1.8 | 353.0 ± 5.6 | 66.3 ± 2.1 | 17.1 ± 3.2^{d} | 249.1 ± 24.5^{c} |
| (100) | | | | | | |
| Cmpd 4A | 101.8 ± 3.5 | 19.2 ± 0.3 | 358.7 ± 6.7 | 67.7 ± 0.5 | 60.8 ± 6.9^{b} | 326.4 ± 12.0^{a,b} |
| (7.2) | | | | | | |
| Cmpd 6A | 86.2 ± 8.8 | 16.1 ± 1.4 | 362.1 ± 15.2 | 68.1 ± 1.6 | 54.8 ± 9.3^{b,c} | 324.6 ± 14.7^{a,b} |
| (2.8) | | | | | | |
| Cmpd 4A | 102.5 ± 13.4 | 19.7 ± 2.6 | 347.7 ± 13.7 | 67.0 ± 1.2 | 29.5 ± 4.1^{c,d} | 271.5 ± 5.8^{b,c} |
| (7.2) + | | | | | | |
| Cmpd 6A | | | | | | |
| (2.8) | | | | | | |

¹Groups not sharing a superscript are significantly different from each other (p < 0.05).

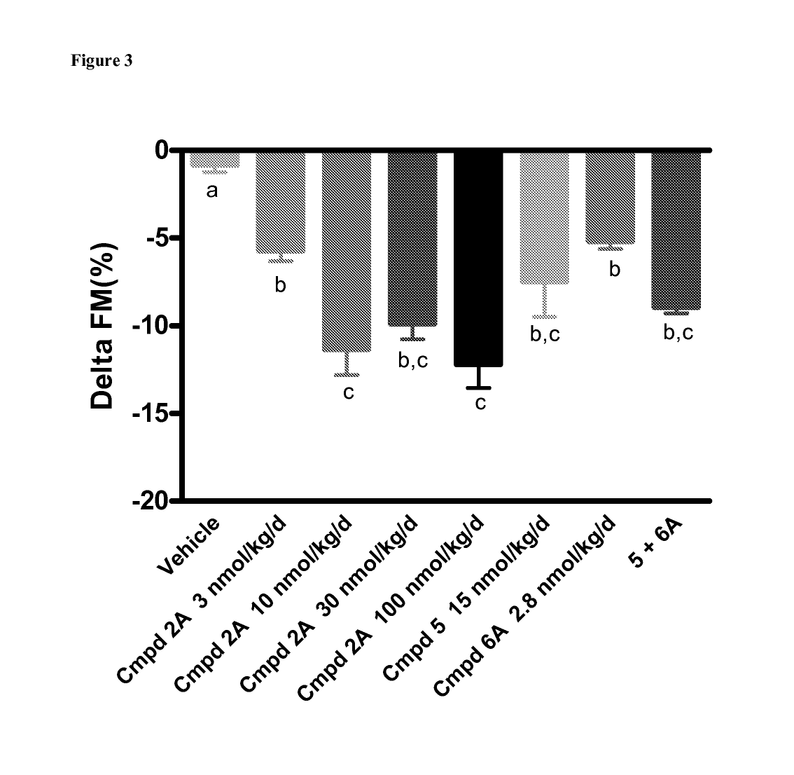

To correct for loss of body weight, reductions in fat and lean mass were adjusted for terminal body weight to calculate the change in adiposity (percent fat mass) and change in percent lean mass. Adiposity was significantly reduced by all doses of Cmpd 2A, as well as the parent compounds alone and in combination (See FIG. 3). Cmpd 2A at 100 nmol/kg/d produced the greatest decrease in adiposity (−12.2±1.3%; FIG. 3).

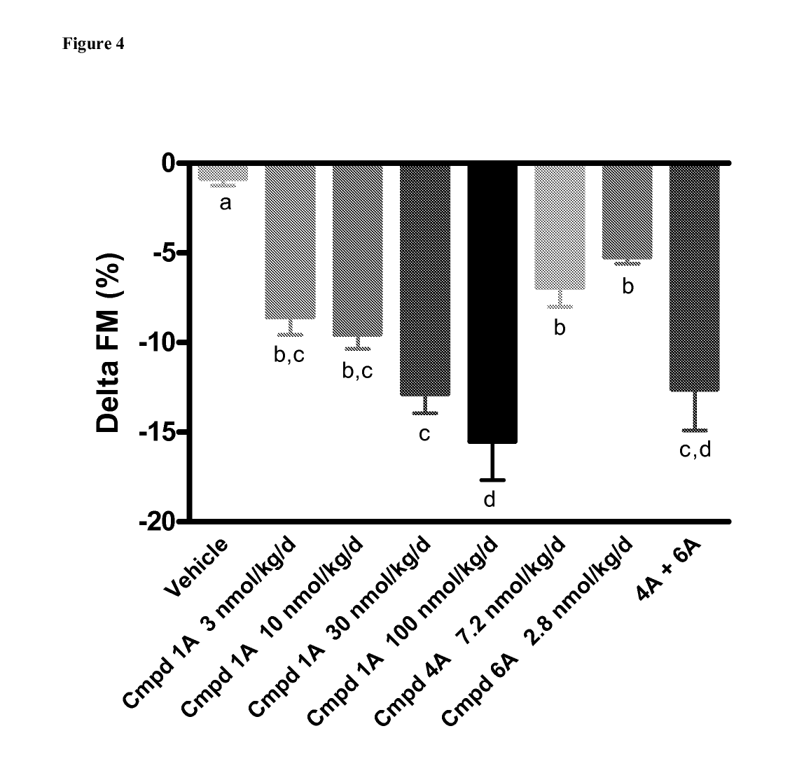

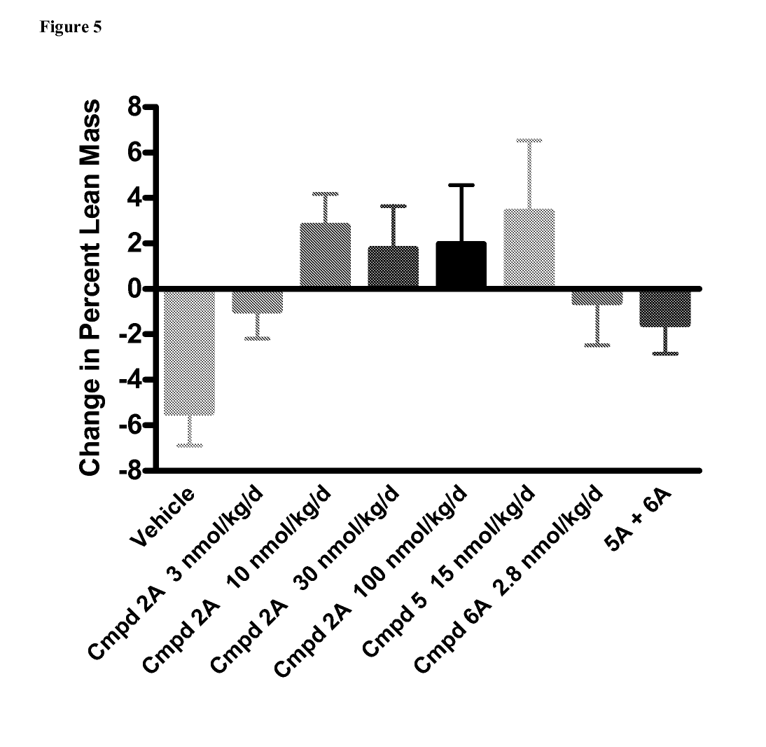

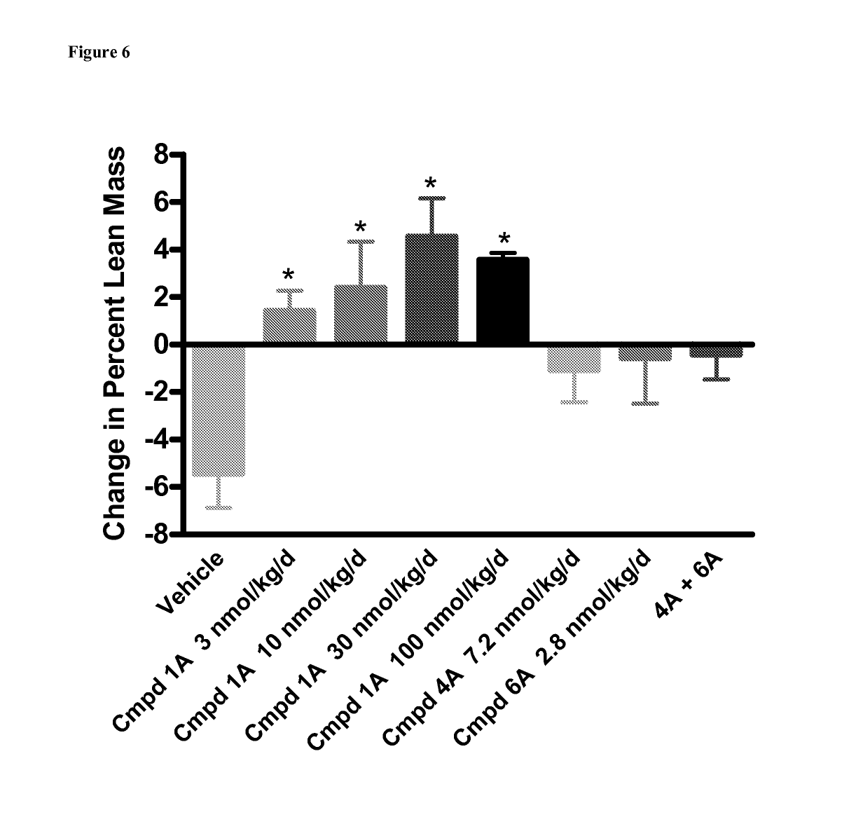

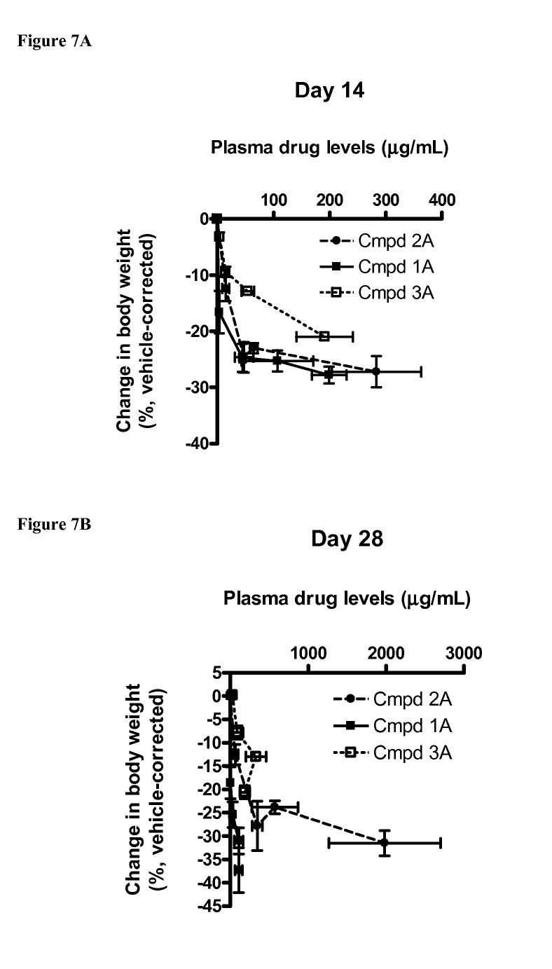

Reductions in adiposity were observed with Cmpd 1A: all treatment doses significantly reduced adiposity compared to vehicle controls, as did Cmpd 4A and Cmpd 6A administration. See FIG. 4. The highest dose of Cmpd 1A produced the greatest decrease (−15.5±2.2%), significantly lower than all other doses, but similar to co-administration of parent compounds (see FIG. 4) and superior to Cmpd 2A.

Whereas overall lean (or fat-free) mass was reduced by high doses of Polypeptide Conjugate treatment, the change in lean mass when expressed as a percent of body weight was not significantly different between vehicle controls and any dose of Cmpd 2A, Cmpd 5, Cmpd 6A or Cmpd 5+Cmpd 6A combination group (See FIG. 5). In surprising contrast, treatment with each dose of Cmpd 1A increased percent lean mass significantly compared to vehicle controls (see FIG. 6). Cmpd 1A is superior in improving body composition, e.g. lean-sparing, fat wasting.

At 28 days of exposure, Compound 1A and 2A provide fasted blood glucose-lowering (mg/dL), HbA1c reduction and body weight loss (fat-wasting, lean-sparing) in ob/ob mice (obese and diabetic). In the ob/ob mouse, Compound 1A provided similar fasted blood glucose-lowering loss relative to exendin-4 but provided superior weight loss (fat-wasting, lean-sparing). These beneficial effects on reducing HbA1c and lowering fasting blood glucose are further surprising in view of the observed increase in HbA1c and increase in fasting blood glucose observed with davalintide alone in this system. This superior effect of Cmpd 1A is even further surprising compared to an increase in plasma glucose levels at day 28 observed in the ob/ob mice treated with a combination of exendin-4 and davalintide (data not shown).

### Example 6

### Compound 1A Provides Superior Metabolic Parameters

Plasma parameters. The effects of the parent and Polypeptide Conjugate compounds on the following plasma parameters were determined: percent hemoglobin A1c (HbA1c), fed state plasma insulin, and concentration of the Polypeptide Conjugate in plasma, at 14 and 28 days.

After 28 days, no change in glucose, percent hemoglobin A1c (HbA1c), insulin, total or HDL cholesterol was observed with Cmpd 2A treatment. Likewise there was no significant effect of Cmpd 1A on total or HDL cholesterol or HbA1c levels after 28 days. Plasma insulin and glucose levels were significantly lowered by Cmpd 1A at some doses compared to vehicle controls. Plasma triglycerides were significantly reduced by all peptide treatments compared to vehicle after 28 days of treatment. Plasma levels of Cmpd 1A and Cmpd 2A, measured by a specific immunoassay, were detected at increasing levels corresponding to treatment dose after both 2 and 4 weeks of treatment.

After 14 days of treatment, HbA1c levels were slightly, but significantly, elevated with Cmpd 5+Cmpd 6A, Cmpd 5 and Cmpd 2A at 10 nmol/kg/d relative to both vehicle and Cmpd 6A groups (See Table 4). No differences in HbA1c were apparent between any of these groups after 28 days. All treatment groups exhibited significantly reduced insulin levels after 14 days relative to vehicle controls, however no differences were observed after 28 days. Plasma levels of Cmpd 2A were significantly increased in plasma in a dose-dependent manner at 14 and 28 days (see Table 4).

TABLE 4
HbA1c, insulin and Cmpd 2A plasma drug levels

| | HbA1c | HbA1c | Insulin | Insulin | Cmpd 2A | Cmpd 2A |
|---|---|---|---|---|---|---|
| Treatment | (%) | (%) | (ng/mL) | (ng/mL) | (ng/mL) | (ng/mL) |
| (nmol/kg/day)¹ | 14 day | 28 day | 14 day | 28 day | 14 day | 28 day |
| Vehicle | 4.6 ± 0.1^{a} | 4.5 ± 0.1 | 5.5 ± 0.8^{a} | 1.7 ± 0.1 | 0^{a} | 0^{a} |
| Cmpd 2A (3) | 4.9 ± 0.1 | 4.6 ± 0.1 | 1.8 ± 0.6^{b} | 2.2 ± 0.9 | 15 ± 6^{a} | 65 ± 35^{a} |
| Cmpd 2A (10) | 4.9 ± 0.1^{b} | 4.6 ± 0.1 | 1.5 ± 0.2^{b} | 1.0 ± 0.2 | 48 ± 16^{a} | 345 ± 67^{a} |
| Cmpd 2A (30) | 4.8 ± 0.1 | 4.6 ± 0.1 | 1.8 ± 0.5^{b} | 0.8 ± 0.1 | 65 ± 7^{a} | 540 ± 232^{a} |
| Cmpd 2A | 4.8 ± 0.1 | 4.5 ± 0.1 | 1.2 ± 0.2^{b} | 0.7 ± 0.1 | 284 ± 79^{b} | 1985 ± 717^{b} |
| (100) | | | | | | |
| Cmpd 5 (15) | 4.9 ± 0.1^{b} | 4.7 ± 0.1 | 1.4 ± 0.2^{b} | 1.5 ± 0.3 | NA | NA |
| Cmpd 6A (2.8) | 4.6 ± 0.1^{a} | 4.6 ± 0.1 | 1.4 ± 0.4^{b} | 1.2 ± 0.2 | NA | NA |
| Cmpd 5 + Cmpd | 4.9 ± 0.1^{b} | 4.8 ± 0.1 | 1.5 ± 0.3^{b} | 1.2 ± 0.3 | NA | NA |
| 6A | | | | | | |

¹Groups not sharing a superscript are significantly different from each other.

In contrast, Cmpd 1A treatment at 3 and 10 nmol/kg/d increased HbA1c levels versus vehicle and Cmpd 6A groups after 14 days, with no differences observed at 28 days (see Table 5). Similar to Cmpd 2A treatment, all doses of Cmpd 1A and the Cmpd 4A+Cmpd 6A combination significantly decreased plasma insulin compared to vehicle at 14 days, but was still reduced further by Cmpd 1A 10 and 30 nmol/kg/d treatment and by Cmpd 4A+Cmpd 6A co-administration treatment relative to vehicle after 28 days (see Table 5). At 14 days Cmpd 1A at 100 nmol/kg/d showed significantly higher plasma concentrations compared to vehicle and to Cmpd 1A at 3 and 10 nmol/kg/d but not 30 nmol/kg/d. There were no differences between 14 day and 28 day values.

TABLE 5
HbA1c, insulin and Cmpd 1A plasma drug levels

| | HbA1c | HbA1c | Insulin | Insulin | Cmpd 1A | Cmpd 1A |
|---|---|---|---|---|---|---|
| Treatment | (%) | (%) | (ng/mL) | (ng/mL) | (ng/mL) | (ng/mL) |
| (nmol/kg/day)¹ | 14 day | 28 day | 14 day | 28 day | 14 day | 28 day |
| Vehicle | 4.6 ± 0.1^{a} | 4.5 ± 0.1 | 5.5 ± 0.8^{a} | 1.7 ± 0.1^{a} | 0^{a} | 0 |
| Cmpd 1A (3) | 4.8 ± 0.1 | 4.7 ± 0.1 | 1.1 ± 0.1^{b} | 0.8 ± 0.1^{a,b} | 3 ± 1^{a} | 3 ± 1 |
| Cmpd 1A (10) | 5.0 ± 0.1^{b} | 4.6 ± 0.1 | 1.2 ± 0.4^{b} | 0.6 ± 0.1^{b} | 24 ± 6^{a} | 30 ± 13 |
| Cmpd 1A (30) | 4.9 ± 0.1^{b} | 4.6 ± 0.1 | 1.0 ± 0.2^{b} | 0.6 ± 0.1^{b} | 107 ± 64^{a,b} | 108 ± 56 |
| Cmpd 1A | 4.8 ± 0.1 | 4.8 ± 0.1 | 1.5 ± 0.5^{b} | 0.8 ± 0.1^{a,b} | 199 ± 31^{b} | 108 ± 46 |
| (100) | | | | | | |
| Cmpd 4A (7.2) | 4.8 ± 0.1 | 4.6 ± 0.1 | 2.6 ± 0.8^{b} | 1.3 ± 0.3^{a} | NA | NA |
| Cmpd 6A (2.8) | 4.6 ± 0.1^{a} | 4.6 ± 0.1 | 1.4 ± 0.4^{b} | 1.2 ± 0.2^{a,b} | NA | NA |
| Cmpd | 4.8 ± 0.1 | 4.6 ± 0.1 | 1.1 ± 0.3^{b} | 0.7 ± 0.1^{b} | NA | NA |
| 4A + Cmpd 6A | | | | | | |

¹Groups not sharing a superscript are significantly different from each other.

This study also evaluated plasma levels of glucose and lipids (triglycerides, total and HDL cholesterol) after 28 days. All doses of Cmpd 2A and parent peptides administered separately or together did not alter glucose, or total or HDL cholesterol. Triglycerides were significantly reduced by all groups compared to vehicle (see Table 6).

TABLE 6
Plasma glucose and lipids: Cmpd 2A

| | Total | HDL | | |
|---|---|---|---|---|
| Treatment | cholesterol | cholesterol | Triglycerides | Glucose |
| (nmol/kg/day) | (mg/dL) | (mg/dL) | (mg/dL) | (mg/dL) |
| Vehicle | 136 ± 7 | 34 ± 1 | 533 ± 24^{a} | 136 ± 4 |
| Cmpd 2A (3) | 116 ± 8 | 33 ± 2 | 295 ± 75^{b} | 132 ± 9 |
| Cmpd 2A (10) | 115 ± 14 | 33 ± 5 | 146 ± 41^{b} | 115 ± 6 |
| Cmpd 2A (30) | 112 ± 4 | 32 ± 1 | 128 ± 25^{b} | 111 ± 2 |
| Cmpd 2A (100) | 107 ± 3 | 30 ± 1 | 139 ± 17^{b} | 110 ± 4 |
| Cmpd 5 (15) | 110 ± 8 | 32 ± 2 | 195 ± 41^{b} | 125 ± 2 |
| Cmpd 6A (2.8) | 142 ± 11 | 39 ± 2 | 223 ± 35^{b} | 126 ± 5 |
| Cmpd 5 + Cmpd 6A | 127 ± 5 | 35 ± 2 | 159 ± 16^{b} | 173 ± 62 |

In contrast to all other compounds including Cmpd 2A, Cmpd 1A decreased HDL cholesterol at 10 and 30 nmol/kg/d, e.g. as compared to Cmpd 6A. Cmpd 1A did not alter total cholesterol. See Table 7. All doses of Cmpd 1A, and parent peptides, decreased triglycerides relative to vehicle, and lowered glucose at 10 and 30 nmol/kg/d, as did Cmpd 4A+Cmpd 6A (see Table 7).

TABLE 7
Plasma glucose and lipids: Cmpd 1A

| | Total | HDL | | |
|---|---|---|---|---|
| Treatment | cholesterol | cholesterol | Triglycerides | Glucose |
| (nmol/kg/day) | (mg/dL) | (mg/dL) | (mg/dL) | (mg/dL) |
| Vehicle | 136 ± 7 | 34 ± 1 | 533 ± 24^{a} | 136 ± 4^{a} |
| Cmpd 1A (3) | 119 ± 4 | 33 ± 1 | 192 ± 39^{b} | 109 ± 3^{b} |
| Cmpd 1A (10) | 108 ± 6 | 29 ± 1^{a} | 121 ± 24^{b} | 111 ± 2^{b} |
| Cmpd 1A (30) | 110 ± 8 | 29 ± 1^{a} | 98 ± 15^{b} | 123 ± 15 |
| Cmpd 1A (100) | 143 ± 15 | 35 ± 1 | 100 ± 9^{b} | 122 ± 8 |
| Cmpd 4A (7.2) | 121 ± 5 | 33 ± 2 | 157 ± 13^{b} | 116 ± 1 |
| Cmpd 6A (2.8) | 142 ± 11 | 39 ± 2^{b} | 223 ± 35^{b} | 126 ± 5 |
| Cmpd 4A ± Cmpd 6A | 120 ± 9 | 33 ± 2 | 142 ± 49^{b} | 107 ± 4^{b} |

### Example 7

### Polypeptide Conjugates Retain Desired Dual-Receptor Agonism

Polypeptide Conjugates Cmpd 1A, Cmpd 2A, Cmpd 3A and parent compounds were tested in a cell-based assay for their ability to bind and agonize the GLP-1 receptor (e.g. exendin-4 like activity) or the calcitonin receptor (e.g. davalintide/amylin mimetic like activity).

The GLP-1 receptor functional assay measures increases in cAMP in the 6-23 (clone 6) cell line (Zeytinoglu et al. “Establishment of a calcitonin-producing rat medullary thyroid carcinoma cell line: I. Morphological studies of the tumor and cells in culture,” Endocrinology 107:509-515 (1980); Crespel et al., “Effects of glucagon and glucagon-like peptide-1-(7-36) amide on C cells from rat thyroid and medullary thyroid carcinoma CA-77 cell line,” Endocrinology 137:3674-3680 (1996)) via the peptide-induced activation of endogenously expressed GLP-1 receptor. Accumulation of cAMP is measured following 30 minute peptide treatment using the HTRF (CisBio, Bedford, Mass. USA) cell-based cAMP assay kit in 384-well format. HTRF (homogeneous time resolved fluorescence) is a technology based on TR-FRET (Time-Resolved Fluorescence and Fluorescence Resonance Energy Transfer), a combination of FRET chemistry and the use of fluorophores with long emission half-lives. Efficacy of test compound is determined relative to cell treatment with 10 microM forskolin (a constitutive activator of adenylate cyclase which leads to cAMP generation), and potency (EC50) of test compound is determined by the analysis of a concentration-response curve using non-linear regression analysis fitted to a 4-parameter model. Concentration-response curves range from 1 micromolar to 0.1 picomolar test compound concentrations (N=4 replicates per concentration). Test compounds are serially diluted 1:10 for an eight-point dose response. Cells are suspended at 2.5×10⁻⁶ cell/ml in buffer containing 500 microM IBMX and HTRF solution. Five microliters of test compound is added to 5 microliters of suspended cells and incubated for 30 minutes in the dark at room temperature. Activation is stopped by addition of detection reagent/lysis buffer. Accumulated cAMP was determined according to the kit instructions.

Functional activity of test compounds at the rat calcitonin C1a receptor was determined by cAMP accumulation in C1a-HEK cell line over-expressing the rat C1a calcitonin receptor, following a 30 minute exposure to test compound using the HTRF-based cAMP assay kit (CisBio, Bedford, Mass. USA) in 384-well format. Accumulation of cAMP is measured in response to increasing concentrations of test peptide and efficacy of said peptide determined relative to cell treatment with 10 microM forskolin (a constitutive activator of adenylate cyclase). Functional activity of a test compound can also be measured using an AlphaScreen Whole Cell cAMP Functional Assay (Perkin Elmer, Mass. USA). Calculated EC50 values are based on 4-parameter concentration-response curves with peptide doses for 1 micromolar to 0.1 picomolar concentrations (N=4 replicates per concentration). Cells are suspended at 2.0×10−6 cell/ml in buffer containing 500 microM IBMX and HTRF solution. Five microliters of test compound is added to 5 microliters of suspended cells and incubated for 30 minutes in the dark at room temperature. Activation is stopped by addition of detection reagent/lysis buffer. Accumulated cAMP was determined according to the kit instructions.

Table 8 provides EC50 measurements for cAMP, an in vitro indicator of receptor activity. As expected exendin-4, Cmpd 4A, Cmpd 5 and Cmpd 10A, all GLP-1 receptor agonists, were active in the GLP-1 receptor function assay but not in the C1a calcitonin receptor function assay. Exendin-4 was more active than any of the other parent compounds. As expected Cmpd 6A, an amylin mimetic davalintide, was active in the C1a calcitonin receptor function assay but not in the GLP-1 receptor function assay.

TABLE 8
Receptor Agonism

| Compound | GLP-1 Function | Calcitonin Function |
|---|---|---|
| number | (nM) | (nM) |
| Cmpd 6A | inactive | 0.05-0.11 |
| Exendin-4 | 0.004 | inactive |
| Cmpd 5 | 0.04 | inactive |
| Cmpd 4A | 0.05 | inactive |
| Cmpd 10A | 0.09 | inactive |
| Cmpd 3A | 0.05 | 2.1 |
| Cmpd 2A | 0.36 | 1.8 |
| Cmpd 1A | 0.19 | 3.2 |

Furthermore, in vivo Compound 1A and 2A have active amylinmimetic sequences based on a demonstrated affect of each compound to lower blood calcium, a property of amylin agonists but not exendin agonists (data not shown). Accordingly, all conjugate compounds Cmpd 1A, Cmpd 2A and Cmpd 3A (a previously known conjugate) were functional at both GLP-1 and amylin receptors.

### Example 8

### Polypeptide Conjugates are Active in Basal Glucose Lowering

Polypeptide Conjugates and parent compounds were analyzed in an in vivo basal glucose lowering assay. This assay reflects the conjugate polypeptide's ability to enhance insulin-mediated glucose clearance of orally administered glucose challenge (Oral Glucose Tolerance Test; OGTT). The OGTT is used to diagnose diabetes, although the simpler fasting plasma glucose test, which measures a subject's plasma glucose level after fasting for at least eight hours, is preferred. The following procedure was used: Test compound at various concentrations was injected intraperitoneally (IP) at t=−5 min to 4-hour fasted NIH/Swiss female mice. Glucose gavage (1.5 g/kg) was given at t=0. Sample was taken at t=30 minutes as tail blood glucose using a OneTouch® Ultra® (LifeScan, Inc., Milpitas, Calif.). Significant effects were identified by ANOVA (p<0.05), followed by Dunnett's post test using GraphPad Prism version 4.00 for Windows (GraphPad Software, San Diego Calif.).

The results are presented in Table 9. As expected exendin-4 and exendin-4 peptide analogs Cmpd 4A, Cmpd 5 and Cmpd 10A, are active in glucose lowering. Cmpd 2A is surprisingly more potent than either previously known Polypeptide Conjugate Cmpd 3A and Cmpd 7A. Cmpd 1A is surprisingly more potent than Cmpd 2A as well as Cmpd 3A and Cmpd 7A.

TABLE 9
OGTT

| Test Compound | ED50 in OGTT (nmol/kg) |
|---|---|
| Cmpd 10A | 3-4 |
| Exendin-4 | 0.4-1 |
| Cmpd 5 | 1.3 |
| Cmpd 4A | 0.7 |
| Cmpd 3A | 6 |
| Cmpd 7A | 4 |
| Cmpd 2A | 1.4 |
| Cmpd 1A | 1.8 |

### Example 9

### Polypeptide Conjugates Cmpd 1A and Cmpd 2A Reduce Food Intake with less Nausea

To investigate possible nausea effects of the conjugate polypeptide, acute kaolin intake was measured in rats. Pica behavior (ingestion of dirt/clay) is a marker of nausea in rodents, typically associated with reduced food intake and weight loss. Pica can be assessed by measuring intake of the synthetic clay kaolin. Cisplatin, a chemotherapy drug that can act in the gut to produce emesis, was used to induce nausea-associated hypophagia as a positive control. Rats were acclimated to kaolin for 3 days as kaolin clay mixed in with regular chow. A 4 hr and a 24 hr baseline kaolin and chow intake were then measured. Subsequently, rats were fasted for approximately 16 hrs after which test compound was injected at the dose indicated below. At 24 hr post injection chow and kaolin consumption was measured. Table 10 presents the results of kaolin intake and correlation to (chow) food intake inhibition.

TABLE 10
Nausea

| Compound | | Inhibited food | Kaolin intake |
|---|---|---|---|
| Number | Dose | intake | (g) |
| Cisplatin | 5 mg/kg Pos. contr. | Yes ~20% | 2.1 |
| Cmpd 7A | 15 nmol/kg/d | Yes ~20% | 2.1 |
| Cmpd 3A | 15 nmol/kg/d | Yes ~20% | 0.5 |
| Cmpd 2A | 30 nmol/kg/d | Yes ~68% | 0.3 |
| Cmpd 1A | 30 nmol/kg/d | Yes ~64% | 0.2 |

Cmpd 7A, a previously known conjugate, at a dose that inhibited food intake, induced significant kaolin intake similar to the positive control, a sign of nausea in rats. Cmpd 3A at doses that suppressed food intake acutely similar to cisplatin injection, had only a modest, if any, effect on kaolin consumption. Cmpd 1A and Cmpd 2A at doses that elicited reductions in food intake even greater than the positive control, Cmpd 3A or 7A, did not induce significant increases in kaolin consumption. Surprisingly, despite nausea associated with exendin-4 and davalintide, Cmpd 1A and Cmpd 2A displayed no significant kaolin intake in this study.

As noted Compound 1A displays a high potency for both the GLP-1 receptor and the amylin and calcitonin receptors, demonstrating that their exendin-like and davalintide moieties retain their biological activities. The activities for these target receptors are only moderately attenuated compared with the parent compounds. Interestingly, Compound 1A binds the CGRP receptor with very low affinity, displaying better selectivity than Compound 3A for amylin receptor (>600 fold versus >100 fold, respectively) and calcitonin receptors (>1600 fold vents >280 fold) against the CGRP receptor, and even better than davalintide (Compound 6A) selectivity for binding to calcitonin and amylin receptors against the CGRP receptor. Despite davalintide being a potent adrenomedullin receptor antagonist (IC50=18 nM), Compound 1A did not display functional activation or antagonism of the adrenomedullin receptor at concentrations up to 10 uM. Accordingly, Compound 1A presents a surprisingly different pharmacological profile compared to davalintide with respect to cellular receptors that recognize amylin and amylinomimetics. Compound 1A has fewer off-target activities than the parent peptide. This improved pharmacological profile for Compound 1A is expected to result in decreased side-effects, such as reduced severe flushing, nausea and/or vomiting, particularly with human subjects, as compared to the parent peptide Compound 6A. For example, CGRP and CGRP agonists have been reported to induce severe flushing, and even nausea and vomiting, in human subjects, which is believed in part due to activation of CGRP receptors and which is relieved by CGRP antagonists.

It is expected that Cmpd 1A and Cmpd 2A, and particularly Compound 1A, will have increased patient compliance and/or allow increased dosing as needed compared to previous compounds, for example compared to Compound 6A, resulting in improved commercial success.

### Example 10

### Polypeptide Conjugates Delay Gastric Emptying

Polypeptide Conjugates and parent compounds were analyzed for their ability to delay gastric emptying in rats. Inhibition of gastric emptying is a physiological effect of GLP-1 receptor agonism as well as amylin receptor agonism, and a key pharmacological effect of exendin-4 and Cmpd 6A in glucose control. Fasted male Sprague Dawley rats (˜250 grams, n=5 per group) received a single subcutaneous injection of saline, Cmpd 6A or test compound at t=0 (1 nmol/kg). Rats then received an oral gavage of 33 mg acetaminophen/1 ml Orablend (Paddock Laboratories, Inc., MN USA) at t=3.5 hr, 5.5 hr or 7.5 hr post-injection. Blood was collected for measurement of acetaminophen at 4 hr, 6 hr or 8 hr after SC injection. Gastric emptying was assessed by the appearance of acetaminophen in plasma 30 min after oral gavage.

Table 11 presents percent inhibition of gastric emptying. Cmpd 1A and Cmpd 2A were as efficacious as Cmpd 6A at inhibiting gastric emptying up to six hours after a single injection. Cmpd 3A and Cmpd 7A did not significantly inhibit gastric emptying at the time point and doses tested. Surprisingly, Cmpd 1A provided a longer duration of action compared to Cmpd 2A.

TABLE 11
Delayed Gastric Emptying

| | 1 nmol/kg, Percent Inhibition | | |
|---|---|---|---|
| Test | of Gastric Emptying | | |
| Compound | 4 hr | 6 hr | 8 hr |
| Cmpd 6A | 75 | 58 | 6 |
| Cmpd 3A | 33 | 0 | 0 |
| Cmpd 2A | 79 | 49 | 4 |
| Cmpd 1A | 48 | 37 | 27 |

The effect of Cmpd 1A, a Polypeptide Conjugate comprising the parent compound Cmpd 4A, in the assays provided herein is surprising. Hargrove et al. 2007 teaches that Cmpd 4A, the exendin-4 peptide analog Leu14 exendin-4, displays a markedly less potent delay of gastric emptying (4 fold less), a markedly less potent inhibition of food intake (8 fold less), a shorter half-life and a shorter duration of action compared to exendin-4. Despite the presence of the Leu14 Exendin-4 peptide analog sequence in the Polypeptide Conjugate Cmpd 1A, Cmpd 1A presents a surprisingly superior pharmacological properties compared to Cmpd 2A and previously known conjugates, such as robust and longer acting inhibition of gastric emptying activity as well as a surprisingly robust and long acting reduction of food intake and reduction in body weight. Surprisingly, Cmpd 1A and Cmpd 2A have a more stable (at least two fold) metabolic profile in vitro in human plasma and with human kidney brush border membrane matrices over a 5 hr incubation period compared to exendin-4, Cmpd 4A and Cmpd 6A (data not shown). No metabolites were detected for Cmpd 1A or Cmpd 2A during that period, which indicates that any unidentified metabolite would have been presents at levels below 10%.

Surprisingly, Cmpd 1A and Cmpd 2A have a longer half-life with similar bioavailability (subcutaneous injection in rats) compared to either exendin-4, Cmpd 4A or Cmpd 6A (data not shown). Compound 1A has a half-life of 72 minutes given subcutaneously (in male Sprague-Dawley rats) compared to exendin-4 of 20 minutes and davalintide of about 30 minutes, with similar absolute bioavailibility.

These superior pharmacological properties, coupled with an excellent PK profile and other favorable drug properties such as fewer off-target activities and reduced nausea, provide a surprisingly useful Polypeptide Conjugate (Cmpd 2A and even more so Cmpd 1A) for controlling glucose with further improvement in controlling body weight and composition in subjects in need of such treatment having diseases and conditions where such treatment is beneficial. Such conditions include subjects having prediabetes, diabetes, diabetes with overweight or obesity, overweight or obesity, where such subjects are in need of and desirous of controlling blood glucose (e.g. anti-hyperglycemia) and/or having improved effect or control of body weight and body composition to reduce body weight, maintain body weight, prevent an increase in body weight and/or improve lean muscle to body fat ratio.

All publications and patent applications are incorporated herein by reference and to the same extent as if each was specifically and individually indicated to be incorporated by reference and as though fully set forth herein. Although the foregoing has been described in detail for purposes of clarity of understanding, it will be apparent to one of ordinary skill in the art that changes and modifications may be made without departing from the spirit or scope of the disclosure or appended claims.

What is claimed is:

1. A Polypeptide Conjugate comprising Compound 1A or Compound 2A.

2. The Polypeptide Conjugate of claim 1 comprising Compound 1A.

3. The Polypeptide Conjugate of claim 1 comprising Compound 2A.

4. The Polypeptide Conjugate of any one of claims 1 to 3 wherein Compound 1A or Compound 2A is covalently linked to at least one polyethylene glycol moiety.

5. The Polypeptide Conjugate of claim 4 wherein the polyethylene glycol is linked to a lipophilic moiety.

6. The Polypeptide Conjugate of claim 5 the lipophilic moiety is an alkyl group, a fatty acid, a cholesteryl, or an adamantyl.

7. The Polypeptide Conjugate compound of any one of claims 1 to 3 wherein Compound 1A or Compound 2A is covalently linked to at least one fatty acid.

8. The Polypeptide Conjugate compound of any one of claims 1 to 3 wherein Compound 1A or Compound 2A is covalently linked to albumin.

9. The Polypeptide Conjugate of claim 8 wherein the albumin is linked to a fatty acid.

10. The Polypeptide Conjugate compound of any one of claims 1 to 3 wherein Compound 1A or Compound 2A is covalently linked to a polyamino acid.

11. The Polypeptide Conjugate of any one of claims 1 to 10 having an EC50 of less than 1 micromolar in a GLP-1 receptor function assay.

12. The Polypeptide Conjugate of claim 11 having an EC50 of less than 100 nanomolar in a GLP-1 receptor function assay.

13. The Polypeptide Conjugate of claim 12 having an EC50 of less than 10 nanomolar in a GLP-1 receptor function assay.

14. The Polypeptide Conjugate of claim 13 having an EC50 of less than 1 nanomolar in a GLP-1 receptor function assay.

15. The Polypeptide Conjugate of any one of claims 1 to 14 wherein the Polypeptide Conjugate has an EC50 of less than 1 micromolar in a calcitonin C1a receptor function assay.

16. The Polypeptide Conjugate of claim 15 wherein the Polypeptide Conjugate has an EC50 of less than 100 nanomolar in a calcitonin C1a receptor function assay.

17. The Polypeptide Conjugate of claim 16 wherein the Polypeptide Conjugate has an EC50 of less than 10 nanomolar in a calcitonin C1a receptor function assay.

18. The Polypeptide Conjugate of claim 17 wherein the Polypeptide Conjugate has an EC50 of less than 5 nanomolar in a calcitonin C1a receptor function assay.

19. The Polypeptide Conjugate of any one of claims 1 to 18 wherein the Polypeptide Conjugate reduces body weight more potently than exendin-4, Cmpd 4A or davalintide.

20. The Polypeptide Conjugate of any one of claims 1 to 19 wherein the Polypeptide Conjugate reduces body weight more potently than both exendin-4 and davalintide.

21. The Polypeptide Conjugate of any one of claims 1 to 20 wherein the Polypeptide Conjugate reduces body weight more efficaciously than exendin-4.

22. The Polypeptide Conjugate of any one of claims 1 to 20 wherein the Polypeptide Conjugate reduces body weight more efficaciously than davalintide.

23. The Polypeptide Conjugate of any one of claims 1 to 22 wherein the Polypeptide Conjugate reduces body weight more efficaciously than both exendin-4 and davalintide.

24. The Polypeptide Conjugate of any one of claims 1 to 20 wherein the Polypeptide Conjugate reduces body weight more efficaciously than co-administered maximally efficacious doses of exendin-4 and davalintide.

25. The Polypeptide Conjugate of any one of claims 1 to 24 wherein the Polypeptide Conjugate reduces body weight more potently and more efficaciously than exendin-4.

26. The Polypeptide Conjugate of any one of claims 1 to 24 wherein the Polypeptide Conjugate reduces body weight more potently and/or more efficaciously than Cmpd 3A.

27. The Polypeptide Conjugate of any one of claims 1 to 26 wherein the reduction in body weight occurs over a period of 4 weeks.

28. The Polypeptide Conjugate of any one of claims 1 to 27 wherein the reduction in body weight occurs over a period of 6 months.

29. The Polypeptide Conjugate of any one of claims 1 to 28 wherein the reduction in body weight occurs over a period of 1 year.

30. The Polypeptide Conjugate of any one claims 1 to 29 wherein the Polypeptide Conjugate has reduced kaolin intake in rats compared to exendin-4.

31. The Polypeptide Conjugate of any one of claims 1 to 30 wherein the Polypeptide Conjugate has reduced kaolin intake in rats compared to davalintide.

32. The Polypeptide Conjugate of any one of claims 1 to 31 wherein the Polypeptide Conjugate has reduced kaolin intake in rats compared to Cmpd 7A.

33. The Polypeptide Conjugate of any one of claims 1 to 32 wherein the Polypeptide Conjugate has reduced nausea compared to exendin-4.

34. The Polypeptide Conjugate of any one of claims 1 to 33 wherein the Polypeptide Conjugate has reduced nausea compared to davalintide.

35. The Polypeptide Conjugate of any one of claims 1 to 34 wherein the Polypeptide Conjugate has reduced nausea compared to Cmpd 7A.

36. The Polypeptide Conjugate of any one of claims 1 to 35 wherein the reduced nausea is a lesser severity of nausea or less frequent number of adverse events per year of nausea or both, wherein the nausea events can be mild, moderate or severe or the combined total number of nausea events.

37. The Polypeptide Conjugate of any one of claims 1 to 36 wherein the Polypeptide Conjugate lowers fasting plasma glucose.

38. The Polypeptide Conjugate of any one of claims 1 to 37 wherein the Polypeptide Conjugate increases tolerance to oral glucose load.

39. The Polypeptide Conjugate of any one of claims 1 to 38 wherein the Polypeptide Conjugate increases glucose-induced insulin secretion.

40. The Polypeptide Conjugate of any one of the claims 1 to 39 wherein the Polypeptide Conjugate delays gastric emptying for at least two, at least four or at least eight hours.

41. The Polypeptide Conjugate of any one of the claims 1 to 40 wherein the Polypeptide Conjugate delays gastric emptying to a greater extent than Cmpd 3A at identical doses.

42. The Polypeptide Conjugate of any one of the claims 1 to 41 wherein the Polypeptide Conjugate lowers plasma triglycerides to a greater extent than exendin-4, davalintide or Cmpd 3A at identical doses.

43. The Polypeptide Conjugate of any one of the claims 1 to 42 wherein the Polypeptide Conjugate lowers plasma triglycerides to a greater extent than Compound 2A at identical doses.

44. The compound of any one of claims 1-43 in the form of a pharmaceutically acceptable salt.

45. A pharmaceutical composition comprising a Polypeptide Conjugate according to any one of claims 1 to 44 and a pharmaceutically acceptable carrier.

46. A method for treating diabetes in a subject in need thereof or desirous thereof comprising administering a therapeutically effective amount of the Polypeptide Conjugate or pharmaceutical composition of any one of claims 1-45 to treat diabetes in the subject.

47. The method of claim 46, wherein the diabetes is Type 1 diabetes.

48. The method of claim 46, wherein the diabetes is Type 2 diabetes.

49. The method of claim 46, wherein the diabetes is gestational diabetes.

50. The method of any one of claims 46 to 49 wherein the subject is overweight, obese or has a tendency to overweight of obese.

51. A method for treating insulin resistance in a subject in need thereof or desirous thereof comprising administering a therapeutically effective amount of the Polypeptide Conjugate or pharmaceutical composition of any one of claims 1-45 to treat insulin resistance in the subject.

52. A method for treating postprandial hyperglycemia in a subject in need thereof or desirous thereof comprising administering a therapeutically effective amount of the Polypeptide Conjugate or pharmaceutical composition of any one of claims 1-45 to treat postprandial hyperglycemia in the subject.

53. A method for lowering blood glucose levels in a subject in need thereof or desirous thereof comprising administering a therapeutically effective amount of the Polypeptide Conjugate or pharmaceutical composition of any one of claims 1-45 to lower blood glucose levels in the subject.

54. A method for lowering HbA1c levels in a subject in need thereof comprising administering a therapeutically effective amount of the Polypeptide Conjugate or pharmaceutical composition of any one of claims 1-45 to lower HbA1c levels in the subject.

55. A method for stimulating insulin release in a subject in need thereof comprising administering a therapeutically effective amount of the Polypeptide Conjugate or pharmaceutical composition of any one of claims 1-45 to stimulate insulin release in the subject.

56. A method for reducing gastric motility in a subject in need thereof comprising administering a therapeutically effective amount of the Polypeptide Conjugate or pharmaceutical composition of any one of claims 1-45 to reduce gastric motility in the subject.

57. A method for delaying gastric emptying in a subject in need thereof comprising administering a therapeutically effective amount of the Polypeptide Conjugate or pharmaceutical composition of any one of claims 1-45 to delay gastric emptying in the subject.

58. A method for reducing food intake in a subject in need or desirous thereof comprising administering a therapeutically effective amount of the Polypeptide Conjugate or pharmaceutical composition of any one of claims 1-45 to reduce food intake in the subject.

59. A method for reducing appetite in a subject in need or desirous thereof comprising administering a therapeutically effective amount of the Polypeptide Conjugate or pharmaceutical composition of any one of claims 1-45 to reduce appetite in the subject.

60. A method for reducing weight in a subject in need or desirous thereof comprising administering a therapeutically effective amount of the Polypeptide Conjugate or pharmaceutical composition of any one of claims 1-45 to reduce weight in the subject.

61. A method for treating overweight in a subject in need thereof or desirous thereof comprising administering a therapeutically effective amount of the Polypeptide Conjugate or pharmaceutical composition of any one of claims 1-45 to treat overweight in the subject.

62. A method for treating obesity in a subject in need thereof or desirous thereof comprising administering a therapeutically effective amount of the Polypeptide Conjugate or pharmaceutical composition of any one of claims 1-45 to treat obesity in the subject.

63. The method of any one of claims 46-62, wherein the therapeutically effective amount of the compound is from about 0.1 μg to about 5 mg.

64. The method of any one of claims 46-62, wherein the therapeutically effective amount of the compound is from about 1 μg to about 2.5 mg.

65. The method of any one of claims 46-62, wherein the therapeutically effective amount of the compound is from about 1 μg to about 1 mg.

66. The method of any one of claims 46-62, wherein the therapeutically effective amount of the compound is from about 1 μg to about 50 μg.

67. The method of any one of claims 46-62, wherein the therapeutically effective amount of the compound is from about 1 μg to about 25 μg.

68. The method of any one of claims 46-62, wherein the therapeutically effective amount of the compound is from about 0.01 μg to about 100 μg based on the weight of a 70 kg subject.

69. The method of any one of claims 46-62, wherein the therapeutically effective amount of the compound is from about 0.01 μg to about 50 μg based on the weight of a 70 kg subject.

70. A drug delivery device comprising at least one therapeutically effective dose of the compound or pharmaceutical composition of any one of claims 1-45.

71. The drug delivery device of claim 70, wherein the drug delivery device is a vial, a pharmaceutical pen, or a cartridge.

72. The drug delivery device of claim 70 or 71, comprising about a one month supply of therapeutically effective doses.

73. A process of making the compound of any one of claims 1 to 45.

74. The process of claim 73 that comprises a recombinant process.

75. A kit comprising the Polypeptide Conjugate or pharmaceutical composition of any one of claims 1 to 45, optionally having instructions for use of the Polypeptide Conjugate or composition by the subject.

76. A method of any one of the above claims wherein administration of the Polypeptide Conjugate results in improved patient compliance compared to Compound 6A, improved reduction in severe flushing compared to Compound 6A, and/or reduced nausea compared to Compound 6A.

77. The method of claim 63 wherein the dose is about 0.1 to 1.0 mg per day.

78. The method of claim 67 wherein the dose is about 0.3 to 0.6 mg per day.

79. The method of any one of the above claims where the dose is given once a day (QD) or split to twice a day (BID).
